(19) United States
(12) Patent Application Publication
(10) Pub. No.: US 2021/0222185 A1
(43) Pub. Date: Jul. 22, 2021

(54) COMPOSITIONS AND METHODS FOR INCREASING EXTRACTABILITY OF SOLIDS FROM COFFEE BEANS

(71) Applicant: Tropic Biosciences UK Limited, Norwich (GB)

(72) Inventors: Eyal MAORI, Cambridge (GB); Yaron GALANTY, Cambridge (GB); Cristina PIGNOCCHI, Norwich (GB); Angela CHAPARRO GARCIA, Norwich (GB); Ofir MEIR, Norfolk (GB)

(73) Assignee: Tropic Biosciences UK Limited, Norwich (GB)

(21) Appl. No.: 16/618,136

(22) PCT Filed: May 31, 2018
(86) PCT No.: PCT/IB2018/053900

§ 371 Date: Nov. 28, 2019

(30) Foreign Application Priority Data
May 31, 2017 (GB) 1708665.3

(51) Int. Cl. C12N 15/82; C12N 9/40; A23F 5/02

(52) U.S. Cl. CPC C12N 15/8243; C12N 15/8213; A23F 5/02; C12Y 302/01022; C12N 9/2465

(57) ABSTRACT

A coffee plant comprising a genome comprising a loss of function mutation in a nucleic acid sequence encoding alpha-D-galactosidase. Also provided is a method of increasing extractability of solids from coffee beans. In addition there is provided a method of producing soluble coffee.

### RELATED APPLICATIONS

Tis application is a National Phase of PCT Patent Application No. PCT/IB2018/053900 having International filing date of May 31, 2018, which claims the benefit of priority of Great Britain Patent Application No. 1708665.3 filed on May 31, 2017. The contents of the above applications are all incorporated by reference as if fully set forth herein in their entirety.

### SEQUENCE LISTING STATEMENT

The ASCII file, entitled 79532SequenceListing.txt, created on Nov. 28, 2019, comprising 33,500 bytes, submitted concurrently with the filing of this application is incorporated herein by reference. The sequence listing submitted herewith is identical to the sequence listing forming part of the international application.

### FIELD AND BACKGROUND OF THE INVENTION

The present invention, in some embodiments thereof, relates to compositions and methods for increasing extractability of solids from coffee beans.

Coffee is a very important agricultural crop with more than 7 million tones of green beans produced every year on about 11 millions hectares. In terms of economic importance, it is second only to oil.

Traditional breeding of coffee is aimed at improving the income of planters, who are mainly small farmers. It is time consuming, due to the biological cycle of the coffee tree. It takes at least 3 years to harvest the first crop of fruits from one progeny and 5 years are necessary for yield evaluation. Two major species, Coffea arabica (self-pollinated, allotetraploid; 2n=44, 68% of global production) and Coffea canephora (self sterile, diploid 2n=22) are cultivated in all tropic areas. Arabica lines are traditionally based on pure line selection and therefore are sensitive to different plant diseases. In arabica, desired traits are primarily pest and disease resistances to introduce into elite varieties. On the other hand, Canephora breeding is more oriented towards improving yield, technological and organoleptic quality through creation of hybrids between genotypes of different genetic groups or selection of improved clones.

As for other perennial crops, coffee has a long juvenile period and conventional breeding for the introduction of new traits, mainly related to resistance or quality, can take between 25-35 years. It is a major drawback for coffee improvement; therefore, genetic engineering could potentially shorten this time period.

However, genetically engineered/modified (GM) crops have been facing increasing disapproval and lack of consumer acceptance because of potential associated risks to the environment and food safety.

Additional background art includes:
EP Pat. EP1436402;
U.S. Pat. Publ. No. 20040199943;
U.S. Pat. No. 6,329,191;
Zhu and Goldstein, Gene 140 (1994), 227-231;
U.S. Pat. No. 7,238,858;
Hoffmann 2017 PlosOne 12(2):e0172630;
Chiang et al., 2016. SP1,2,3. Sci Rep. 2016 Apr. 15; 6:24356.

### SUMMARY OF THE INVENTION

According to an aspect of some embodiments of the present invention there is provided a coffee plant comprising a genome comprising a loss of function mutation in a nucleic acid sequence encoding alpha-D-galactosidase.

According to an aspect of some embodiments of the present invention there is provided a method of increasing extractability of solids from coffee beans, the method comprising:

(a) subjecting a coffee plant cell to a DNA editing agent directed at a nucleic acid sequence encoding alpha-D-galactosidase to result in a loss of function mutation in the nucleic acid sequence encoding the alpha-D-galactosidase; and
(b) regenerating a plant from the plant cell.

According to some embodiments of the invention, the method further comprises harvesting beans from the plant.

According to some embodiments of the invention, the mutation is in a homozygous form.

According to some embodiments of the invention, the mutation is in a heterozygous form.

According to an aspect of some embodiments of the present invention there is provided the plant as described herein or ancestor thereof having been treated with a DNA editing agent directed to the genomic sequence encoding alpha-D-galactosidase.

According to some embodiments of the invention, the mutation is selected from the group consisting of a deletion, an insertion an insertion/deletion (Indel) and a substitution.

According to some embodiments of the invention, the coffee plant is from a species Coffea arabica.

According to some embodiments of the invention, the coffee plant is from a species Coffea canephora.

According to some embodiments of the invention, the subjecting is to a nucleic acid construct encoding the DNA editing agent.

According to some embodiments of the invention, the subjecting is by a DNA-free delivery method.

According to an aspect of some embodiments of the present invention there is provided a nucleic acid construct comprising a nucleic acid sequence encoding a DNA editing agent directed at coffee alpha-D-galactosidase being operably linked to a plant promoter.

According to some embodiments of the invention, the DNA editing agent is of a DNA editing system selected from the group consisting of selected from the group consisting of meganucleases, Zinc finger nucleases (ZFNs), transcription-activator like effector nucleases (TALENs) and CRISPR-Cas.

According to some embodiments of the invention, the DNA editing agent is of a DNA editing system comprising CRISPR-Cas.

According to some embodiments of the invention, the nucleic acid sequence encoding alpha-D-galactosidase is as set forth in SEQ ID NO: 4.

According to some embodiments of the invention, the nucleic acid sequence encoding alpha-D-galactosidase is selected from the group consisting of SEQ ID NOs: 2-4.

According to some embodiments of the invention, the nucleic acid sequence encoding alpha-D-galactosidase is as set forth in SEQ ID NO: 2.

According to some embodiments of the invention, the nucleic acid sequence encoding alpha-D-galactosidase is as set forth in SEQ ID NO: 3.

According to some embodiments of the invention, the DNA editing agent is directed at nucleic acid coordinates within exon 1, 2, 3, 4 and/or 5 of a nucleic acid sequence encoding the alpha-D-galactosidase.

According to some embodiments of the invention, the DNA editing agent comprises a nucleic acid sequence at least 99% identical to a nucleic acid sequence selected from the group consisting of SEQ ID NO: 38-41.

According to some embodiments of the invention, the DNA editing agent comprises a nucleic acid sequence at least 99% identical to a nucleic acid sequence selected from the group consisting of SEQ ID NO: 9-11 and 37

According to some embodiments of the invention, the DNA editing agent comprises a nucleic acid sequence selected from the group consisting of SEQ ID NO: 38-41.

According to some embodiments of the invention, the DNA editing agent comprises a plurality of nucleic acid sequences selected from the group consisting of SEQ ID NOs: 9-11 and 37.

According to some embodiments of the invention, the DNA editing agent is directed to a plurality of alpha-D-galactosidase genes.

According to some embodiments of the invention, the plurality of alpha-D-galactosidase genes are selected from the group consisting of SEQ ID NOs: 2-4.

According to some embodiments of the invention, the plurality of alpha-D-galactosidase genes are selected from the group consisting of SEQ ID NOs: 3-4.

According to some embodiments of the invention, the plurality of alpha-D-galactosidase genes are selected from the group consisting of SEQ ID NOs: 1-2.

According to some embodiments of the invention, the plurality of alpha-D-galactosidase genes are selected from the group consisting of SEQ ID NOs: 1 and 3.

According to an aspect of some embodiments of the present invention there is provided a plant part of the plant as described herein.

According to some embodiments of the invention, the plant part is a bean.

According to some embodiments of the invention, the bean is dry.

According to an aspect of some embodiments of the present invention there is provided a method of producing coffee beans, the method comprising:

(a) growing the plant as described herein; and
(b) harvesting beans from the plant.

According to an aspect of some embodiments of the present invention there is provided a method of producing soluble coffee, the method comprising subjecting beans as described herein to extraction, dehydration and optionally roasting.

According to an aspect of some embodiments of the present invention there is provided soluble coffee of the beans as described herein.

According to some embodiments of the invention, the soluble coffee is in a powder form.

According to some embodiments of the invention, the soluble coffee is in a granulated form.

According to some embodiments of the invention, the soluble coffee is decaffeinated.

According to some embodiments of the invention, the soluble coffee comprises DNA of the beans.

According to some embodiments of the invention, the plant is non-transgenic.

According to an aspect of some embodiments of the present invention there is provided a coffee plant, or part thereof, comprising a loss of function mutation introduced into a genomic nucleic acid sequence encoding alpha-D-galactosidase protein, wherein the mutation results in a reduced level or reduced activity of the protein as compared to a coffee plant lacking the loss of function mutation.

According to some embodiments of the invention, the plant, or part thereof, comprises one or more non-natural loss of function mutations introduced into one or more genomic nucleic acid sequences encoding one or more alpha-D-galactosidase proteins, wherein said one or more mutations each results in reduced levels or reduced activities of the proteins as compared to a coffee plant lacking the loss of function mutation.

According to some embodiments of the invention, the non-natural loss of function mutation was introduced using a DNA editing agent.

According to some embodiments of the invention, the plant does not comprise a transgene encoding the DNA editing agent, a transgene encoding a selectable marker or a reporter, or does not comprising a transgene encoding any of the DNA editing agent, the selectable marker, or the reporter.

According to some embodiments of the invention, the DNA editing agent comprised a DNA editing system selected from the group consisting of meganucleases, Zinc finger nucleases (ZFNs), transcription-activator like effector nucleases (TALENs) and CRISPR-Cas.

According to some embodiments of the invention, the DNA editing agent was CRISPR-Cas.

According to some embodiments of the invention, the mutation is homozygous.

According to some embodiments of the invention, the mutation is selected from the group consisting of a deletion, an insertion, an insertion/deletion (Indel), and a substitution.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawings will be provided by the Office upon request and payment of the necessary fee. Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart of an embodiment of the method of selecting cells comprising a genome editing event;

FIG. 2 shows the quantification of genome editing activity in coffee protoplasts using a reporter sensor and FACS according to FIG. 1. Protoplasts were transfected with different versions of the sensor construct (1 to 4) each expressing GFP+mCherry and different sgRNAs targeting GFP. Positive editing of the GFP marker was evaluated by measuring the reduction of the GFP signal compared to the control without sgRNA. 4 days after transfection, cells were analysed for efficient genome editing by measuring the ratio of green versus red protoplasts. Genome-editing of the GFP sensor was measured by the reduction of the green/red protoplasts ratio. All sensor constructs with specific sgRNAs targeting GFP showed a reduction of green versus red when compared to the control plasmid in coffee protoplasts.

FIGS. 3A-C show the identification of alpha-D-galactosiade gene(s) for targeting in coffee. The alpha D galactosidase genes within the coffee genome were identified by blasting the gene with the accession number AJ887712.1 from Marraccini et al., 2005 Plant Physiol Biochem. 2005 October-November; 43(10-11):909-20. FIG. 3A shows the result from the blast search: 3 complete genes of alpha D galactosidase were found within the genome (SEQ ID NOs: 2-4). FIG. 3B shows a percentage identity matrix to the AJ887712.1 of the identified genes. FIG. 3C shows the RPKM data of each gene from the coffee genome database.

FIGS. 4A-E show the characterization and genome-editing analysis of the coffee gene alpha-D-galactosidase Cc04_g14280. (FIG. 4A) is a cartoon illustrating the major features of the gene: numbered yellow boxes indicate the exons, Forward and Reverse arrows represent primers used for amplification of the target area, and sgRNA1 and sgRNA2 indicate the sites along the gene where the sgRNAs were designed. (FIG. 4B) Cc04_g14280 was amplified with primers “Forward” (TCCAGTCCTACTTTATGATTGAAAA, SEQ ID NO: 42) and “Reverse2” (TTTCCTTGGGGCTTATGTTG, SEQ ID NO: 43)), which flank the sgRNA1 and sgRNA2 region as depicted in FIG. 4A, using DNA extracted 6 days post transfection from coffee transfected and sorted protoplasts as template (FIG. 4B). Samples were transfected with the following plasmids: (1) pDK2029 (control, no sgRNAs), (2) pDK2030 [sgRNA1 (SEQ ID NO: 9) and sgRNA2 (SEQ ID NO: 10) targeting Cc04_g14280 (SEQ ID NO: 4) as depicted in FIG. 4A] and (3) PCR negative control (no DNA). The agarose gel indicates a deletion has occurred in the target gene of around 250 bp. (FIG. 4C) shows an alignment of the cloned PCR products in FIG. 4B columns 1 and 2. The sequence from PCR product 1 was the same as WT, while all 5 colonies from PCR product 2 showed a deletion of 239 bp situated between the two sgRNA target sites, 3 bp upstream from the PAM site (blue arrows pointing to red circles). (FIG. 4D) is the longest peptide sequence of a 6-frame translation of the clones 1 (from samples transfected with plasmid pDK2029 non-targeting sgRNA) and 2 (from samples transfected with plasmid pDK2030). The 239 bp deletion induced an early stop codon as indicated by the red box. (FIG. 4E) is an amino acid alignment of the two peptide sequences in FIG. 4D clearly showing the 239 bp deletion.

FIGS. 5A-C show the characterization and genome-editing analysis of the coffee putative alpha-D-galactosidase gene Cc02_g05490. (FIG. 5A) is a cartoon illustrating the major features of the gene: yellow boxes represent the exons, horizontal arrows represent primers used for amplification of the target area, and sgRNA171 and sgRNA172 indicate the sites along the gene where the sgRNAs were designed. (FIG. 5B) Nested PCR was used to amplify Cc02_g05490 with primers 118 to 121, which flank the sgRNA171 and sgRNA172 region as depicted in FIG. 5A, using DNA extracted 6 days post transfection from coffee transfected and sorted protoplasts as template. Samples were transfected with the following plasmids: (1) pDK2031 (control, sgRNAs uniquely targeting Cc04_g4280), (2) pDK2032 [sgRNA171 (SEQ ID NO: 10) targeting Cc02_g05490 (SEQ ID NO: 3) as depicted in FIG. 5A], (3) pDK2033 [sgRNA172 (SEQ ID NO: 41) targeting Cc02_g05490 (SEQ ID NO: 3) and (4) PCR negative control (no DNA). The agarose gel shows the amplification of the targeted region. (FIG. 5C) shows an alignment of the cloned PCR products in FIG. 5B columns 2 and 3 where several small indels are shown. The position of the sgRNAs are shown within a green oval.

FIGS. 6A-C show the characterization and genome-editing analysis of the coffee putative alpha-D-galactosidase gene Cc11_g00330. (FIG. 6A) is a cartoon illustrating the major features of the gene: yellow boxes represent the exons, horizontal arrows represent primers used for amplification of the target area, and sgRNA169 and sgRNA170 indicate the sites along the gene where the sgRNAs were designed. (FIG. 6B) Nested PCR was used to amplify Cc11_g00330 with primers 114 to 117, which flank the sgRNA169 and sgRNA170 region as depicted in panel A, using DNA extracted at 6 days post transfection from coffee transfected and sorted protoplasts as template. Samples were transfected with the following plasmids: (1) pDK2031 (control, sgRNAs uniquely targeting Cc04_g14280), (2) pDK2032 [sgRNA169 (SEQ ID NO: 38) targeting Cc11_g00330 (SEQ ID NO: 2) as depicted in FIG. 6A], (3) pDK2033 [sgRNA170 (SEQ ID NO: 39) targeting Cc11_g00330 (SEQ ID NO: 2) and (4) PCR negative control (no DNA). The agarose gel shows the amplification of the targeted region. (FIG. 6C) shows an alignment of the cloned PCR products in FIG. 6B column 3 where several small indels are shown. The position of the sgRNA is shown within a green oval.

FIGS. 7A-E Coffee protoplasts regeneration. FIG. 7A. Freshly isolated coffee protoplasts; FIG. 7B. First cell divisions occur 48 h after protoplast isolation; FIG. 7C. Microcalli of embryogenic cells develop after 2 months; FIG. 7D. Embryogenic calli of 1-2 mm develop from microcalli; FIG. 7E. Embryo development from embryogenic cells (red square).

FIGS. 8A-B show the regeneration of transfected coffee protoplasts. FIG. 8A. Embryogenic calli obtained from transfected protoplasts three months post-transfection were transferred to regeneration medium containing MS salts and vitamins; FIG. 8B. First embryos were regenerated after 3-4 weeks.

FIGS. 9A-C show the sequences of α-D-galactosidase genes, sgRNA binding sites and sgRNA sequences according to some embodiments of the invention. Red highlight denotes the positions of the sgRNAs along the targeted sequences; Grey highlight shows the PAM sequence; Dark Green highlight denotes allelic variation; and Light Green letters denotes the exons.

### DESCRIPTION OF SPECIFIC EMBODIMENTS OF THE INVENTION

The present invention, in some embodiments thereof, relates to compositions and methods for increasing extractability of solids from coffee beans.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

In green coffee beans, the polysaccharide fraction represents half the total weight. Among these polysaccharides, mannans account for 50%. Mannans consists of a β-linked mannan chain that can be substituted with galactose residues to give galactomannans. The ratio of mannans to galactomannans affects the water solubility of the polymer. The more galactomannans to mannans present, the more soluble the polymer is. The activity of the enzyme α-D-galactosidase in coffee has been reported to be responsible in the removal of galactose residues from galactomannans forming mannans, reducing the water solubility of the polymer.

Embodiments described herein relate to inhibition of α-D-galactosidase expression at the genome level so as to increase the water soluble fraction from coffee beans. The gene encoding α-D-galactosidase has therefore been targeted for genome modification by the genome editing system, CRISPR-Cas9.

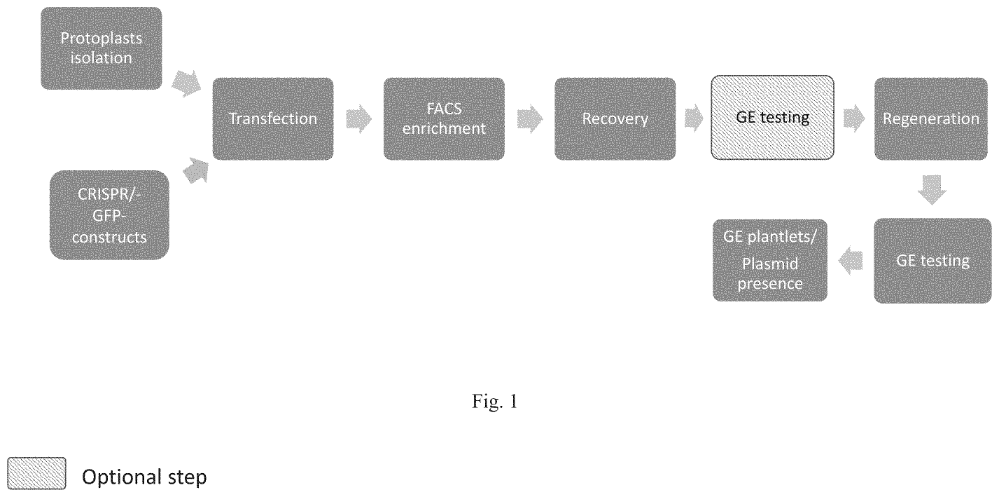

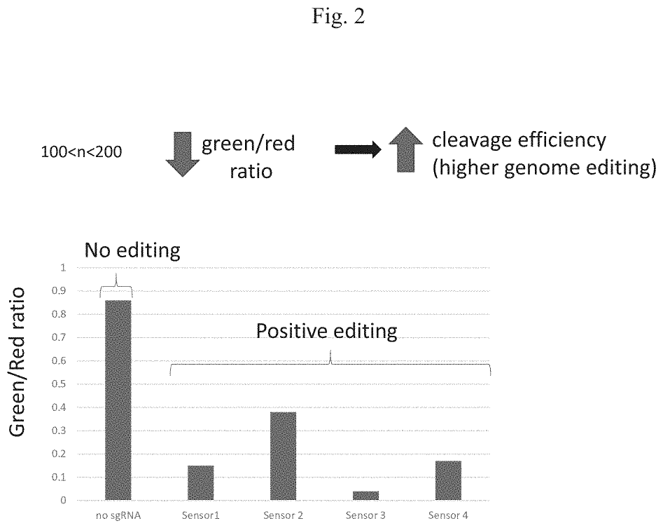

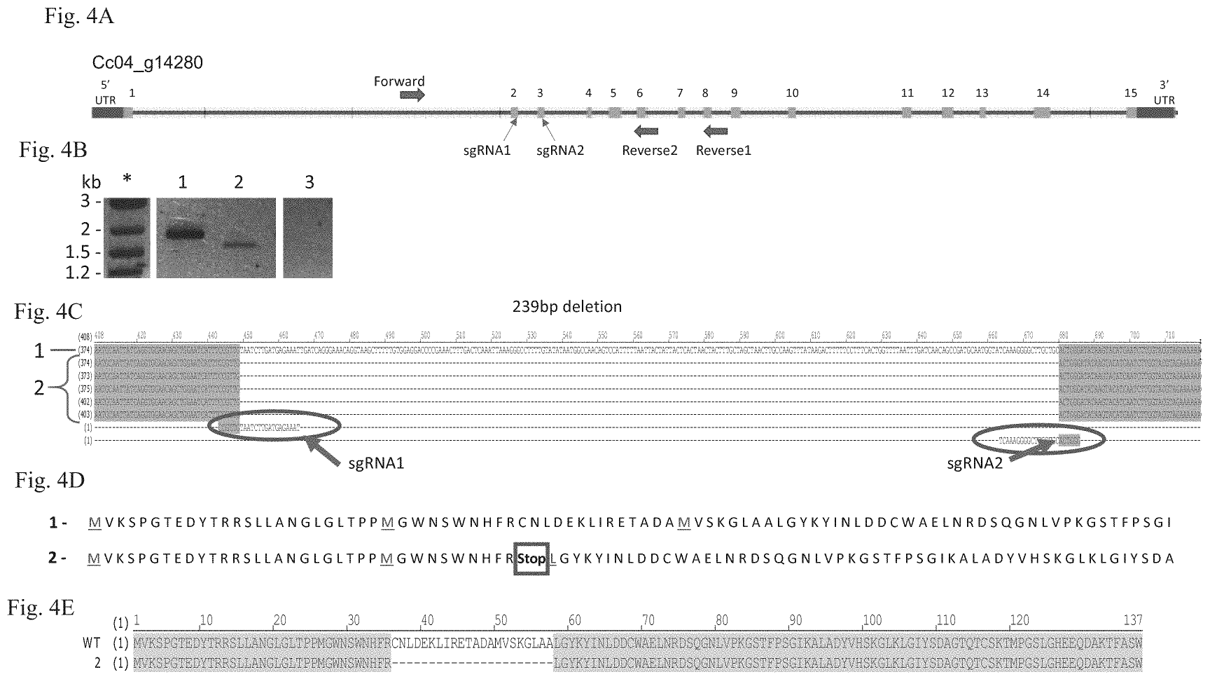

As is illustrated herein, the present inventors have established a genome editing system in coffee protroplasts, followed by selection that results in non-transgenic protoplasts that can be efficiently regenerated into a coffee plant (see FIGS. 1 and 2). The present inventors have further identified three α-D-galactosidase genes as targets for editing, two of them being remote homologs of less than 80% identity to AJ887712.1 from Marraccini et al., 2005, supra. Expression analysis revealed a biologically relevant pattern of expression especially for Cc04_g14280 that emphasizes their role in the removal of galactose residues from galactomannans in coffee beans. All three genes were targeted individually or simultaneously to result in non-transgenic genome editing (see FIGS. 4A-E to 6A-C) that results in loss of expression of these genes in coffee beans. Protoplasts comprising these genome editing events were subjected to regeneration so as to result in mature plants.

Hence, present results show for the first time non-transgenic genome editing of α-D-galactosidase genes in coffee, which can be harnessed to increase the water soluble fraction from coffee beans.

Thus, according to an aspect of the invention there is provided a method of modifying a genome of a coffee plant cell or plant, the method comprising subjecting a genome of the coffee cell or plant to a DNA editing agent so as to induce a loss of function mutation(s) in at least one allele of a α-D-galactosidase gene in the genome of the coffee.

As used herein a “coffee” refers to a plant of the family Rubiaceae, genus Coffea. There are many coffee species. Embodiments of the invention may refer to two primary commercial coffee species: Coffea arabica (C. arabica), which is known as arabica coffee, and Coffea canephora, which is known as robusta coffee (C. robusta). Coffea liberica Bull. ex Hiern is also contemplated here which makes up 3% of the world coffee bean market. Also known as Coffea arnoldiana De Wild or more commonly as Liberian coffee. Coffees from the species arabica are also generally called “Brazils” or they are classified as “other milds”. Brazilian coffees come from Brazil and “other milds” are grown in other high-grade coffee producing countries, which are generally recognized as including Colombia, Guatemala, Sumatra, Indonesia, Costa Rica, Mexico, United States (Hawaii), El Salvador, Peru, Kenya, Ethiopia and Jamaica. Coffea canephora, i.e. robusta, is typically used as a low-cost extender for arabica coffees. These robusta coffees are typically grown in the lower regions of West and Central Africa, India, Southeast Asia, Indonesia, and also Brazil. A person skilled in the art will appreciate that a geographical area refers to a coffee growing region where the coffee growing process utilizes identical coffee seedlings and where the growing environment is similar.

As used herein “plant” refers to whole plant(s), a grafted plant, ancestors and progeny of the plants and plant parts, including seeds, fruits, shoots, stems, roots (including tubers), rootstock, scion, and plant cells, tissues and organs.

According to a specific embodiment, the plant part is a bean.

“Grain,” “seed,” or “bean,” refers to a flowering plant's unit of reproduction, capable of developing into another such plant. As used herein, especially with respect to coffee plants, the terms are used synonymously and interchangeably.

According to a specific embodiment, the cell is a germ cell.

According to a specific embodiment, the cell is a somatic cell.

The plant may be in any form including suspension cultures, protoplasts, embryos, meristematic regions, callus tissue, leaves, gametophytes, sporophytes, pollen, and microspores.

According to a specific embodiment, the plant part comprises DNA.

According to a specific embodiment, the coffee plant is of a coffee breeding line, more preferably an elite line.

According to a specific embodiment, the coffee plant is of an elite line.

According to a specific embodiment, the coffee plant is of a purebred line.

According to a specific embodiment, the coffee plant is of a coffee variety or breeding germplasm.

The term “breeding line”, as used herein, refers to a line of a cultivated coffee having commercially valuable or agronomically desirable characteristics, as opposed to wild varieties or landraces. The term includes reference to an elite breeding line or elite line, which represents an essentially homozygous, usually inbred, line of plants used to produce commercial F₁ hybrids. An elite breeding line is obtained by breeding and selection for superior agronomic performance comprising a multitude of agronomically desirable traits. An elite plant is any plant from an elite line. Superior agronomic performance refers to a desired combination of agronomically desirable traits as defined herein, wherein it is desirable that the majority, preferably all of the agronomically desirable traits are improved in the elite breeding line as compared to a non-elite breeding line. Elite breeding lines are essentially homozygous and are preferably inbred lines.

The term “elite line”, as used herein, refers to any line that has resulted from breeding and selection for superior agronomic performance. An elite line preferably is a line that has multiple, preferably at least 3, 4 5, 6 or more (genes for) desirable agronomic traits as defined herein.

The terms “cultivar” and “variety” are used interchangeable herein and denote a plant with has deliberately been developed by breeding, e.g., crossing and selection, for the purpose of being commercialized, e.g., used by farmers and growers, to produce agricultural products for own consumption or for commercialization. The term “breeding germplasm” denotes a plant having a biological status other than a “wild” status, which “wild” status indicates the original non-cultivated, or natural state of a plant or accession.

The term “breeding germplasm” includes, but is not limited to, semi-natural, semi-wild, weedy, traditional cultivar, landrace, breeding material, research material, breeder's line, synthetic population, hybrid, founder stock/base population, inbred line (parent of hybrid cultivar), segregating population, mutant/genetic stock, market class and advanced/improved cultivar. As used herein, the terms “purebred”, “pure inbred” or “inbred” are interchangeable and refer to a substantially homozygous plant or plant line obtained by repeated selfing and-or backcrossing.

A non-comprehensive list, of coffee varieties is provided herein: Wild Coffee: Tis is the common name of “Coffea racemosa Lour” which is a coffee species native to Ethiopia.

Baron Goto Red: A coffee bean cultivar that is very similar to ‘Catuai Red’. It is grown at several sites in Hawaii.

Blue Mountain: Coffea arabica L. ‘Blue Mountain’. Also known commonly as Jamaican coffea or Kenyan coffea. It is a famous arabica cultivar that originated in Jamaica but is now grown in Hawaii, PNG and Kenya. It is a superb coffee with a high quality cup flavor. It is characterized by a nutty aroma, bright acidity and a unique beef-bullion like flavor.

Bourbon: Coffea arabica L. ‘Bourbon’. A botanical variety or cultivar of Coffea arabica which was first cultivated on the French controlled island of Bourbon, now called Réunion, located east of Madagascar in the Indian ocean.

Brazilian Coffea: Coffea arabica L. ‘Mundo Novo’. The common name used to identify the coffee plant cross created from the “Bourbon” and “typica” varieties.

Caracol/Caracoli: Taken from the Spanish word Caracolillo meaning ‘seashell’ and describes the peaberry coffee bean.

Catimor: Is a coffee bean cultivar cross-developed between the strains of Caturra and Hibrido de Timor in Portugal in 1959. It is resistant to coffee leaf rust (Hemileia vastatrix). Newer cultivar selection with excellent yield but average quality.

Catuai: Is a cross between the Mundo Novo and the Caturra arabica cultivars. Known for its high yield and is characterized by either yellow (Coffea arabica L. ‘Catuai Amarelo’) or red cherries (Coffea arabica L. ‘Catuai Vermelho’).

Caturra: A relatively recently developed sub-variety of the Coffea arabica species that generally matures more quickly, gives greater yields, and is more disease resistant than the traditional “old arabica” varieties like Bourbon and typica.

Columbiana: A cultivar originating in Columbia. It is vigorous, heavy producer but average cup quality.

Congencis: Coffea Congencis—Coffee bean cultivar from the banks of Congo, it produces a good quality coffee but it is of low yield. Not suitable for commercial cultivation

DewevreiIt: Coffea DewevreiIt. A coffee bean cultivar discovered growing naturally in the forests of the Belgian Congo. Not considered suitable for commercial cultivation.

DybowskiiIt: Coffea DybowskiiIt. This coffee bean cultivar comes from the group of Eucoffea of inter-tropical Africa. Not considered suitable for commercial cultivation

Excelsa: Coffea Excelsa—A coffee bean cultivar discovered in 1904. Possesses natural resistance to diseases and delivers a high yield. Once aged it can deliver an odorous and pleasant taste, similar to var. arabica.

Guadalupe: A cultivar of Coffea arabica that is currently being evaluated in Hawaii.

Guatemala(n): A cultivar of Coffea arabica that is being evaluated in other parts of Hawaii.

Hibrido de Timor: This is a cultivar that is a natural hybrid of arabica and robusta. It resembles arabica coffee in that it has 44 chromosomes.

Icatu: A cultivar which mixes the “arabica & robusta hybrids” to the arabica cultivars of Mundo Novo and Caturra.

Interspecific Hybrids: Hybrids of the coffee plant species and include; ICATU (Brazil; cross of Bourbon/MN & robusta), S2828 (India; cross of arabica & Liberia), Arabusta (Ivory Coast; cross of arabica & robusta).

‘K7’, ‘SL6’, ‘SL26’, ‘H66”, ‘KP532’: Promising new cultivars that are more resistant to the different variants of coffee plant disease like Hemileia.

Kent: A cultivar of the arabica coffee bean that was originally developed in Mysore India and grown in East Africa. It is a high yielding plant that is resistant to the “coffee rust” decease but is very susceptible to coffee berry disease. It is being replaced gradually by the more resistant cultivar's of ‘S.288’, ‘S.333’ and ‘S.795’.

Kouillou: Name of a Coffea canephora (robusta) variety whose name comes from a river in Gabon in Madagascar.

Laurina: A drought resistant cultivar possessing a good quality cup but with only fair yields.

Maragogipe/Maragogype: Coffea arabica L. ‘Maragopipe’. Also known as “Elephant Bean”. A mutant variety of Coffea arabica (typica) which was first discovered (1884) in Maragogype County in the Bahia state of Brazil.

Mauritiana: Coffea Mauritiana. A coffee bean cultivar that creates a bitter cup. Not considered suitable for commercial cultivation

Mundo Novo: A natural hybrid originating in Brazil as a cross between the varieties of ‘arabica’ and ‘Bourbon’. It is a very vigorous plant that grows well at 3,500 to 5,500 feet (1,070 m to 1,525 m), is resistant to disease and has a high production yield. Tends to mature later than other cultivars.

Neo-Arnoldiana: Coffea Neo-Arnoldiana is a coffee bean cultivar that is grown in some parts of the Congo because of its high yield. It is not considered suitable for commercial cultivation.

Nganda: Coffea canephora Pierre ex A. Froehner ‘Nganda’. Where the upright form of the coffee plant Coffea Canephora is called robusta its spreading version is also known as Nganda or Kouillou.

Paca: Created by El Salvador's agricultural scientists, this cultivar of arabica is shorter and higher yielding than Bourbon but many believe it to be of an inferior cup in spite of its popularity in Latin America.

Pacamara: An arabica cultivar created by crossing the low yield large bean variety Maragogipe with the higher yielding Paca. Developed in El Salvador in the 1960's this bean is about 75% larger than the average coffee bean.

Pache Colis: An arabica cultivar being a cross between the cultivars Caturra and Pache comum. Originally found growing on a Guatemala farm in Mataquescuintla.

Pache Comum: A cultivar mutation of typica (arabica) developed in Santa Rosa Guatemala. It adapts well and is noted for its smooth and somewhat flat cup

Preanger: A coffee plant cultivar currently being evaluated in Hawaii.

Pretoria: A coffee plant cultivar currently being evaluated in Hawaii.

Purpurescens: A coffee plant cultivar that is characterized by its unusual purple leaves.

Racemosa: Coffea Racemosa—A coffee bean cultivar that looses its leaves during the dry season and re-grows them at the start of the rainy season. It is generally rated as poor tasting and not suitable for commercial cultivation.

Ruiru 11: Is a new dwarf hybrid which was developed at the Coffee Research Station at Ruiru in Kenya and launched on to the market in 1985. Ruiru 11 is resistant to both coffee berry disease and to coffee leaf rust. It is also high yielding and suitable for planting at twice the normal density.

San Ramon: Coffea arabica L. ‘San Ramon’. It is a dwarf variety of arabica var typica. A small stature tree that is wind tolerant, high yield and drought resistant.

Tico: A cultivar of Coffea arabica grown in Central America.

Timor Hybrid: A variety of coffee tree that was found in Timor in 1940s and is a natural occurring cross between the arabica and robusta species.

Typica: The correct botanical name is Coffea arabica L. ‘typica’. It is a coffee variety of Coffea arabica that is native to Ethiopia. Var typica is the oldest and most well known of all the coffee varieties and still constitutes the bulk of the world's coffee production. Some of the best Latin-American coffees are from the typica stock. The limits of its low yield production are made up for in its excellent cup.

Villalobos: A cultivar of Coffea arabica that originated from the cultivar ‘San Ramon’ and has been successfully planted in Costa Rica.

As used herein “modifying a genome” refers to introducing at least one mutation in at least one allele of an α-D-galactosidase gene of the coffee. According to some embodiments, modifying refers to introducing a mutation in each allele of the α-D-galactosidase gene of the coffee. According to at least some embodiments, the mutation on the two alleles of the α-D-galactosidase gene is in a homozygous form.

According to some embodiments, mutations on the two alleles of the α-D-galactosidase gene are noncomplementary.

As used herein “α-D-galactosidase gene” refers to the gene encoding the α-D-galactosidase enzyme as set forth in EC 3.2.1.22. For example, the enzymes produced from the genes Cc11_g00330 (SEQ ID NO: 2), Cc02_g05490 (SEQ ID NO: 3) and Cc04_g14280 (SEQ ID NO: 4) present in C. canephora similar to accession number AJ877912 (SEQ ID NO: 5) and in C. arabica to accession number AJ877911 (SEQ ID NO: 6).

According to a specific embodiment, the α-D-galactosidase gene is Cc04_g14280 (SEQ ID NO: 4).

Exemplary sgRNA sequences and alternatively combinations thereof are provided in Table A below.

| | | |
|---|---|---|
| pDK2030 | 122 (SEQ ID NO: 9), | Cc04_g14280/SEQ ID NO: 4 |
| | 123 (SEQ ID NO: 10) | |
| pDK2031 | 124 (SEQ ID NO: 11), | Cc04_g14280/(SEQ ID NO: 4,) |
| | 126 (SEQ ID NO: 37) | |
| pDK2032 | 122 (SEQ ID NO: 9), | Cc04_g14280/(SEQ ID NO: 4), |
| | 123 (SEQ ID NO: 10), | Cc11_g00330/(SEQ ID NO: 2), |
| | 169 (SEQ ID NO: 38), | Cc02_g05490/(SEQ ID NO: 3) |
| | 171 (SEQ ID NO: 40) | |
| pDK2033 | 124 (SEQ ID NO: 11), | Cc04_g14280 (SEQ ID NO: 4), |
| | 126 (SEQ ID NO: 37), | Cc11_g00330/(SEQ ID NO: 2), |
| | 170 (SEQ ID NO: 39), | Cc02_g05490/(SEQ ID NO: 3) |
| | 172 (SEQ ID NO: 41) | |

Also contemplated are naturally occurring functional homologs of each of the above genes e.g., exhibiting at least 80%, 81%, 82%, 83%, 84%, 85%, 86%, 87%, 88%, 89%, 90%, 91%, 92%, 93%, 94%, 95%, 96%, 97% 98% or 99% identity to the above-mentioned genes and having an -alpha-D-galactosidase activity, as defined above.

As used herein, “sequence identity” or “identity” or grammatical equivalents as used herein in the context of two nucleic acid or polypeptide sequences includes reference to the residues in the two sequences which are the same when aligned. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are considered to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Henikoff S and Henikoff JG. [Amino acid substitution matrices from protein blocks. Proc. Natl. Acad. Sci. U.S.A. 1992, 89(22): 10915-9].

Identity can be determined using any homology comparison software, including for example, the BlastN software of the National Center of Biotechnology Information (NCBI) such as by using default parameters.

According to some embodiments of the invention, the identity is a global identity, i.e., an identity over the entire nucleic acid sequences of the invention and not over portions thereof.

α-D-galactosidase enzyme is capable of releasing α-1,6-linked galactose units from galactomannans stored in plant seed storage tissue or maturation. In other words, α-D-galatosidases activity has the capacity to remove galactose residues, that are α-1,6-linked to galactomannan polysaccharides, which brings about a decreased solubility of the polymers.

According to a specific embodiment, the DNA editing agent modifies the target sequence α-D-galactosidase and is devoid of “off target” activity, i.e., does not modify other sequences in the coffee genome.

According to a specific embodiment, the DNA editing agent comprises an “off target activity” on a non-essential gene in the coffee genome.

Non-essential refers to a gene that when modified with the DNA editing agent does not affect the phenotype of the target genome in an agriculturally valuable manner (e.g., caffeine content, flavor, biomass, yield, biotic/abiotic stress tolerance and the like).

Off-target effects can be assayed using methods which are well known in the art and are described herein.

As used herein “loss of function” mutation refers to a genomic aberration which results in reduced ability (i.e., impaired function) or inability of α-D-galactosidase to hydrolyze α-1,6-linked galactose units from insoluble mannans. As used herein “reduced ability” refers to reduced α-D-galactosidase activity (i.e., hydrolysis of α-1,6-linked galactose units, mannan branching) as compared to that of the wild-type enzyme devoid of the loss of function mutation. According to a specific embodiment, the reduced activity is by at least 5%, 10%, 20%, 30%, 40%, 50%, 60%, 70%, 80%, 90% or even more as compared to that of the wild-type enzyme under the same assay conditions. α-Gal activity can be detected spectrophotometrically with the substrate p-nitrophenyl-α-D-galactopyranoside (pNGP). According to a specific embodiment, the reaction mixture contains 200 μl pNGP 100 mM in McIlvain's buffer (citric acid 100 mM-Na₂HPO₄ 200 mM, pH 6.5) up to 1 ml final volume, with enzyme extract as required. The reaction is maintained at 26° C. and started with the addition of enzyme. One volume of reaction mixture is added to 4 volumes of stop solution (Na₂CO₃—NaHCO₃ 100 mM, pH 10.2) and absorption is read at λ=405 nm. Appearance of nitrophenyl is calculated using molar extinction coefficient ε=18300 (specific for pH 10.2) and converted to nkat mg⁻¹ protein (Marracini et al. 2005. Biochemical and molecular characterization of α-D-galactosidase from coffee beans, Plant Physiology and Biochemistry, 43: 909-920)

According to a specific embodiment, the loss of function mutation results in no expression of the α-D-galactosidase mRNA or protein.

According to a specific embodiment, the loss of function mutation results in expression of an α-D-galactosidase protein which is not capable of supporting mannan branching.

According to a specific embodiment, the loss of function mutation is selected from the group consisting of a deletion, insertion, insertion-deletion (Indel), inversion, substitution and a combination of same (e.g., deletion and substitution e.g., deletions and SNPs).

According to a specific embodiment, the loss of function mutation is smaller than 1 Kb or 0.1 Kb.

According to a specific embodiment, the “loss-of-function” mutation is in the 5′ of α-D-galactosidase gene so as to cause a frameshift in the coding sequence which disrupts the production of any functional α-D-galactosidase peptide. Alternatively, and as an example, the mutation may cause a premature stop codon or a nonsense mutation resulting in no expression of the protein.

According to a specific embodiment, the “loss-of-function” mutation is anywhere in the α-D-galactosidase gene that allows the production of an α-D-galactosidase expression product (e.g., first exon), while being unable to facilitate (contribute to) mannan branching i.e., inactive protein or a protein with an impaired catalytic activity, as described above. Also provided herein is a mutation in regulatory elements of the gene e.g., promoter, splice sites and the line.

Examples of suggested positions within Cc04_g14280:

sgRNA Pair 1-Exon 1
(SEQ ID NO: 7)
GGTGAAGTCTCCAGGAACCGAGG;
(SEQ ID NO: 8)
GCTTGGTCTAACACCTCCGATGG;
sgRNA Pair 2-Across Exon2 and 3
ATTTCTCATCAAGATTACAACGG (exon2) (SEQ ID NO: 9,
also referred to as sgRNA 122);
TCAAAGGGGCTTGCTGCACTGGG (exon3) (SEQ ID NO: 10,
also referred to as sgRNA 123);
Pair 3-Exon 5
GATGGGAATGTTGAACCTTTAGG (SEQ ID NO: 11, also
referred to as sgRNA 124);
(SEQ ID NO: 12)
CAGAGTAAATTCCAAGCTTTAGG;

According to a specific embodiment, the DNA editing agent comprises a nucleic acid sequence at least 99% identical to a nucleic acid sequence selected from the group consisting of SEQ ID NO: 38, 39, 40 and 41 (169, 170, 171, 172).

According to a specific embodiment, the DNA editing agent comprises a nucleic acid sequence at least 99% identical to a nucleic acid sequence selected from the group consisting of SEQ ID NO: 38, 39, 40 and 41(169-172).

According to a specific embodiment, the DNA editing agent comprises a nucleic acid sequence at least 99% identical to a nucleic acid sequence selected from the group consisting of SEQ ID NO: 9-11 and 37

According to a specific embodiment, the DNA editing agent comprises a nucleic acid sequence selected from the group consisting of SEQ ID NO: 38-41.

According to a specific embodiment, the DNA editing agent comprises a plurality of nucleic acid sequences selected from the group consisting of SEQ ID NOs: 9-11 and 37.

As mentioned, the coffee plant comprises the loss of function mutation in at least one allele of the α-D-galactosidase gene.

According to a specific embodiment, the mutation is homozygous.

According to a specific embodiment, the mutation is heterozygous.

According to an aspect, there is provided a method of increasing extractability of solids from coffee beans, the method comprising:

(a) subjecting a coffee plant cell to a DNA editing agent directed at a nucleic acid sequence encoding alpha-D-galactosidase to result in an impaired or loss of function mutation in said nucleic acid sequence encoding said alpha-D-galactosidase; and
(b) regenerating a plant from said plant cell.

According to a specific embodiment, the method further comprises harvesting beans from said plant.

Examples of extractable solids which are contemplated herein are provided in Tables 1-2 below, some of them are water extractable.

TABLE 1
Composition of green and roasted coffees (according to variety) and
of instant coffee (expressed as a percentage of the dry basis)

| | Arabica | | Robusta | | |
|---|---|---|---|---|---|
| Component | Green | Roasted | Green | Roasted | Instant |
| Minerals | 3.0-4.2 | 3.5-4.5 | 4.0-4.5 | 4.6-5.0 | 9.0-10.0 |
| Caffeine | 0.9-1.2 | appr. 1.0 | 1.6-2.4 | appr. 2.0 | 4.5-6.1 |
| Trigonelline | 1.0-1.2 | 0.5-1.0 | 0.6-0.75 | 0.3-0.6 | — |
| Lipids | 12.0-18.0 | 14.5-20.0 | 9.0-13.0 | 11.0-16.0 | 1.5-1.6 |
| Total chlorogenic acids | 5.5-8.0 | 1.2-2.3 | 7.0-10.0 | 3.9-4.6 | 5.2-7.4 |
| Aliphatic acids | 1.5-2.0 | 1.0-1.5 | 1.5-2.0 | 1.0-1.5 | — |
| Oligosaccharides | 6.0-8.0 | 0-3.5 | 5.0-7.0 | 0-3.5 | 0.7-1.2 |
| Total polysaccharides | 50.0-55.0 | 24.0-39.0 | 37.0-47.0 | — | appr. 6.5 |
| Amino acids | 2.0 | 0 | 2.0 | 0 | 0 |
| Proteins | 11.0-13.0 | 13.0-15.0 | 11.0-13.0 | 13.0-15.0 | 16.0-21.0 |
| Humic acids | — | 16.0-17.0 | — | 16.0-17.0 | 15.0 |

Data from Clifford [symbol] [symbol]
indicates data missing or illegible when filed

TABLE 2
Composition of green and roasted coffee according to
variety (expressed as a percentage of the dry basis)

| | Arabica | | Robusta | | |
|---|---|---|---|---|---|
| Component | Green (a) | Roasted (b) | Green (a) | Roasted (b) | Instant (b) |
| Humidity | 5-13 | 1-3 | 5-13 | 1-3 | 2-4 |
| Alkaloids (caffeine) | 0.8-1.4 | 1.0-1.6 | 1.7-4.0 | 1.2-2.6 | 2.5-5.0 |
| Trigonelline | 0.6-1.2 | 0.1-1.2 | 0.3-0.9 | 0.1-1.2 | 0.9-1.7 |
| Total glucides | 5.5-66.5 | 16.2-37.5 | 40-55.5 | 16.2-37.5 | 19.3-55.6 |
| Soluble glucides | 6-12.5 | 6.2-16.5 | 6-12.5 | 6.2-16.5 | 1.3-8.6 |
| | holocellulose (c) | | holocellulose (c) | | |
| Insoluble glucides | 34-53 | 10-21 | 34-53 | 10-21 | — |
| Acids | 8-11 | 1.2-7.1 | 9-14 | 1.2-7.1 | — |
| Chlorogenic acids | 7-9 | 0.2-3.5 | 7-12 | 0.2-3.5 | 2.0-4.0 |
| Aliphatic acids | 1-3 | 1.8-4.6 | 1-2 | 1.8-4.6 | 3.5-10.8 |
| Proteins, amino acids | 9-13 | 13-15 | 9-13 | 13-15 | 16-21 |
| Lipids | 15-18 | 15.5-20 | 8-12 | 8.3-13.5 | 0-0.5 |
| Ash | 3.5-4 | 3.5-6 | 3.5-4 | 2.5-6 | 9-10 |
| Volatile aromas | — | Trace | — | Trace | Trace |
| | | amounts | | amounts | amounts |
| Humic acids | — | 16-17 | — | 16-17 | 15 |

(a) From [symbol] [symbol]
indicates data missing or illegible when filed

As used herein “extractability of solids” refers to at least 10%, 20%, 30%, 40%, 50%, 60%, 70%, 80%, 85%, 90% or even 95%, increase solid extractability from beans of a coffee plant having the loss of function mutation in the genome as compared to that of a coffee plant of the same genetic background not comprising the loss of function mutation as assayed by methods which are well known in the art (see Examples section which follows).

For example, solubility can be determined by measuring galactomannans. An increase in galactomannans content is an indication of increased galacomannans to mannas ratio, therefore increased solubility. Galactomannans can be measured indirectly by sequential enzymatic reactions involving β-mannanase, α-galactosidase and β-galactose dehydrogenase and release of D-galactonic acid and NADH. The release of NADH is assayed spectrophotometrically at 340 nm.

Following is a description of various non-limiting examples of methods and DNA editing agents used to introduce nucleic acid alterations to a gene of interest and agents for implementing same that can be used according to specific embodiments of the present disclosure.

Genome Editing using engineered endonucleases—this approach refers to a reverse genetics method using artificially engineered nucleases to typically cut and create specific double-stranded breaks at a desired location(s) in the genome, which are then repaired by cellular endogenous processes such as, homologous recombination (HR) or non-homologous end-joining (NHEJ). NHEJ directly joins the DNA ends in a double-stranded break, while HR utilizes a homologous donor sequence as a template (i.e the sister chromatid formed during S-phase) for regenerating the missing DNA sequence at the break site. In order to introduce specific nucleotide modifications to the genomic DNA, a donor DNA repair template containing the desired sequence must be present during HR (exogenously provided single stranded or double stranded DNA).

Genome editing cannot be performed using traditional restriction endonucleases since most restriction enzymes recognize a few base pairs on the DNA as their target and these sequences often will be found in many locations across the genome resulting in multiple cuts which are not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

Meganucleases—Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location.

This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence.

Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs—Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is FokI. Additionally FokI has the advantage of requiring dimerization to have nuclease activity and this means the specificity increases dramatically as each nuclease partner recognizes a unique DNA sequence. To enhance this effect, FokI nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the FokI domains heterodimerize to create a double-stranded break. Repair of these double-stranded breaks through the non-homologous end-joining (NHEJ) pathway often results in small deletions or small sequence insertions. Since each repair made by NHEJ is unique, the use of a single nuclease pair can produce an allelic series with a range of different deletions at the target site.

In general NHEJ is relatively accurate (about 85% of DSBs in human cells are repaired by NHEJ within about 30 min from detection) in gene editing erroneous NHEJ is relied upon as when the repair is accurate the nuclease will keep cutting until the repair product is mutagenic and the recognition/cut site/PAM motif is gone/mutated or that the transiently introduced nuclease is no longer present.

The deletions typically range anywhere from a few base pairs to a few hundred base pairs in length, but larger deletions have been successfully generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homologous recombination (HR) to generate specific modifications (Li et al., 2011; Miller et al., 2010; Umov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers are typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, Calif.).

Method for designing and obtaining TALENs are described in e.g. Reyon et al. Nature Biotechnology 2012 May; 30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www(dot)talendesign(dot)org). TALEN can also be designed and obtained commercially from e.g., Sangamo Biosciences™ (Richmond, Calif.).

T-GEE system (TargetGene's Genome Editing Engine)—A programmable nucleoprotein molecular complex containing a polypeptide moiety and a specificity conferring nucleic acid (SCNA) which assembles in-vivo, in a target cell, and is capable of interacting with the predetermined target nucleic acid sequence is provided. The programmable nucleoprotein molecular complex is capable of specifically modifying and/or editing a target site within the target nucleic acid sequence and/or modifying the function of the target nucleic acid sequence. Nucleoprotein composition comprises (a) polynucleotide molecule encoding a chimeric polypeptide and comprising (i) a functional domain capable of modifying the target site, and (ii) a linking domain that is capable of interacting with a specificity conferring nucleic acid, and (b) specificity conferring nucleic acid (SCNA) comprising (i) a nucleotide sequence complementary to a region of the target nucleic acid flanking the target site, and (ii) a recognition region capable of specifically attaching to the linking domain of the polypeptide. The composition enables modifying a predetermined nucleic acid sequence target precisely, reliably and cost-effectively with high specificity and binding capabilities of molecular complex to the target nucleic acid through base-pairing of specificity-conferring nucleic acid and a target nucleic acid. The composition is less genotoxic, modular in their assembly, utilize single platform without customization, practical for independent use outside of specialized core-facilities, and has shorter development time frame and reduced costs.

CRISPR-Cas system (also referred to herein as “CRISPR”)—Many bacteria and archaea contain endogenous RNA-based adaptive immune systems that can degrade nucleic acids of invading phages and plasmids. These systems consist of clustered regularly interspaced short palindromic repeat (CRISPR) nucleotide sequences that produce RNA components and CRISPR associated (Cas) genes that encode protein components. The CRISPR RNAs (crRNAs) contain short stretches of homology to the DNA of specific viruses and plasmids and act as guides to direct Cas nucleases to degrade the complementary nucleic acids of the corresponding pathogen. Studies of the type II CRISPR/Cas system of Streptococcus pyogenes have shown that three components form an RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821.).

It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of Cas9 in conjunction with synthetic gRNAs can be used to produce targeted double-stranded brakes in a variety of different species (Cho et al., 2013; Cong et al., 2013; DiCarlo et al., 2013; Hwang et al., 2013a,b; Jinek et al., 2013; Mali et al., 2013).

The CRIPSR/Cas system for genome editing contains two distinct components: a gRNA and an endonuclease e.g. Cas9.

The gRNA is typically a 20 nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break. Just as with ZFNs and TALENs, the double-stranded breaks produced by CRISPR/Cas can be repaired by HR (homologous recombination) or NHEJ (non-homologous end-joining) and are susceptible to specific sequence modification during DNA repair.

The Cas9 nuclease has two functional domains: RuvC and HNH, each cutting a different DNA strand. When both of these domains are active, the Cas9 causes double strand breaks in the genomic DNA.

A significant advantage of CRISPR/Cas is that the high efficiency of this system coupled with the ability to easily create synthetic gRNAs. This creates a system that can be readily modified to target modifications at different genomic sites and/or to target different modifications at the same site. Additionally, protocols have been established which enable simultaneous targeting of multiple genes. The majority of cells carrying the mutation present biallelic mutations in the targeted genes.

However, apparent flexibility in the base-pairing interactions between the gRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is mostly repaired by single strand break repair mechanism involving proteins such as but not only, PARP (sensor) and XRCC1/LIG III complex (ligation). If a single strand break (SSB) is generated by topoisomerase I poisons or by drugs that trap PARP1 on naturally occurring SSBs then these could persist and when the cell enters into S-phase and the replication fork encounter such SSBs they will become single ended DSBs which can only be repaired by HR. However, two proximal, opposite strand nicks introduced by a Cas9 nickase are treated as a double-strand break, in what is often referred to as a ‘double nick’ CRISPR system. A double-nick which is basically non-parallel DSB can be repaired like other DSBs by HR or NHEJ depending on the desired effect on the gene target and the presence of a donor sequence and the cell cycle stage (HR is of much lower abundance and can only occur in S and G2 stages of the cell cycle). Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that are not likely to change the genomic DNA, even though these events are not impossible.

Modified versions of the Cas9 enzyme containing two inactive catalytic domains (dead Cas9, or dCas9) have no nuclease activity while still able to bind to DNA based on gRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

There are a number of publically available tools available to help choose and/or design target sequences as well as lists of bioinformatically determined unique gRNAs for different genes in different species such as the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

Non-limiting examples of a gRNA that can be used in the present disclosure include those described in the Example section which follows.

In order to use the CRISPR system, both gRNA and Cas9 should be in a target cell or delivered as a ribonucleoprotein complex. The insertion vector can contain both cassettes on a single plasmid or the cassettes are expressed from two separate plasmids. CRISPR plasmids are commercially available such as the px330 plasmid from Addgene. Use of clustered regularly interspaced short palindromic repeats (CRISPR)-associated (Cas)-guide RNA technology and a Cas endonuclease for modifying plant genomes are also at least disclosed by Svitashev et al., 2015, Plant Physiology, 169 (2): 931-945; Kumar and Jain, 2015, J Exp Bot 66: 47-57; and in U.S. Patent Application Publication No. 20150082478, which is specifically incorporated herein by reference in its entirety.

“Hit and run” or “in-out”—involves a two-step recombination procedure. In the first step, an insertion-type vector containing a dual positive/negative selectable marker cassette is used to introduce the desired sequence alteration. The insertion vector contains a single continuous region of homology to the targeted locus and is modified to carry the mutation of interest. This targeting construct is linearized with a restriction enzyme at a one site within the region of homology, introduced into the cells, and positive selection is performed to isolate homologous recombination events. The DNA carrying the homologous sequence can be provided as a plasmid, single or double stranded oligo These homologous recombinants contain a local duplication that is separated by intervening vector sequence, including the selection cassette. In the second step, targeted clones are subjected to negative selection to identify cells that have lost the selection cassette via intrachromosomal recombination between the duplicated sequences. The local recombination event removes the duplication and, depending on the site of recombination, the allele either retains the introduced mutation or reverts to wild type. The end result is the introduction of the desired modification without the retention of any exogenous sequences.

The “double-replacement” or “tag and exchange” strategy—involves a two-step selection procedure similar to the hit and run approach, but requires the use of two different targeting constructs. In the first step, a standard targeting vector with 3′ and 5′ homology arms is used to insert a dual positive/negative selectable cassette near the location where the mutation is to be introduced. After the system component have been introduced to the cell and positive selection applied, HR events could be identified. Next, a second targeting vector that contains a region of homology with the desired mutation is introduced into targeted clones, and negative selection is applied to remove the selection cassette and introduce the mutation. The final allele contains the desired mutation while eliminating unwanted exogenous sequences.

Site-Specific Recombinases—The Cre recombinase derived from the P1 bacteriophage and Flp recombinase derived from the yeast Saccharomyces cerevisiae are site-specific DNA recombinases each recognizing a unique 34 base pair DNA sequence (termed “Lox” and “FRT”, respectively) and sequences that are flanked with either Lox sites or FRT sites can be readily removed via site-specific recombination upon expression of Cre or Flp recombinase, respectively. For example, the Lox sequence is composed of an asymmetric eight base pair spacer region flanked by 13 base pair inverted repeats. Cre recombines the 34 base pair lox DNA sequence by binding to the 13 base pair inverted repeats and catalyzing strand cleavage and re-ligation within the spacer region. The staggered DNA cuts made by Cre in the spacer region are separated by 6 base pairs to give an overlap region that acts as a homology sensor to ensure that only recombination sites having the same overlap region recombine.

Basically, the site specific recombinase system offers means for the removal of selection cassettes after homologous recombination events. This system also allows for the generation of conditional altered alleles that can be inactivated or activated in a temporal or tissue-specific manner. Of note, the Cre and Flp recombinases leave behind a Lox or FRT “scar” of 34 base pairs. The Lox or FRT sites that remain are typically left behind in an intron or 3′ UTR of the modified locus, and current evidence suggests that these sites usually do not interfere significantly with gene function.

Thus, Cre/Lox and Flp/FRT recombination involves introduction of a targeting vector with 3′ and 5′ homology arms containing the mutation of interest, two Lox or FRT sequences and typically a selectable cassette placed between the two Lox or FRT sequences. Positive selection is applied and homologous recombination events that contain targeted mutation are identified. Transient expression of Cre or Flp in conjunction with negative selection results in the excision of the selection cassette and selects for cells where the cassette has been lost. The final targeted allele contains the Lox or FRT scar of exogenous sequences.

According to a specific embodiment, the DNA editing agent is CRISPR-Cas9.

Exemplary gRNA sequences are provided in:

Cc04_g14280
(SEQ ID NO: 13)
GGTGAAGTCTCCAGGAACCG;
(SEQ ID NO: 14)
GCTTGGTCTAACACCTCCGA;

The DNA editing agent is typically introduced into the plant cell using expression vectors.

Thus, according to an aspect of the invention there is provided a nucleic acid construct comprising a nucleic acid sequence coding for a DNA editing agent capable of hybridizing to an α-D-galactosidase gene of a coffee and facilitating editing of said α-D-galactosidase gene, said nucleic acid sequence being operably linked to a cis-acting regulatory element for expressing said DNA editing agent in a cell of a coffee.

It will be appreciated that the present teachings also relate to introducing the DNA editing agent using DNA-free methods such as mRNA+gRNA transfection or RNP transfection.

Embodiments of the invention relate to any DNA editing agent, such as described above.

According to a specific embodiment, the genome editing agent comprises an endonuclease, which may comprise or have an auxiliary unit of a DNA targeting module (e.g., sgRNA, or also as referred to herein as “gRNA”).

According to a specific embodiment, the DNA editing agent is CRISPR/Cas9 sgRNA.

According to a specific embodiment, the DNA editing agent is TALEN.

For example, in order to design the TAL Effector to target the alpha-D-Galactosidase, TAL Effector Nucleotides Targeter 2.0, a web-based tool as part of the TAL Effector Nucleotide Targeter (TALE-NT) suite (tale-nt(dot)cac(dot)comell(dot)edu) is used. Exemplary of specificity profiling of TALENs targeting the alpha-D-Galactosidase Cc04_g14280. Sequences are provided in an ideal so TALEN would specifically bind only its intended target sequence and have no off-target activity, thus allowing the targeted cleavage of only a single sequence, e.g Cc04_g14280 allele of a gene in the context of a whole genome. Following are non-limiting examples of Talen sequences that can be used to target the gene according to embodiments of the invention.

TABLE 3

| | | | | | Target |
|---|---|---|---|---|---|
| | | | | | sequence |
| | | | | | (SEQ ID |
| Sequence | TAL | TAL | | | NOs: |
| name | start | length | RVD sequence | Strand | 15-36) |
| AJ877912.1 | 10 | 23 | HD NG HD HD NI | Plus | TCTCCAGG |
| | | | NH NH NI NI HD | | AACCGAGG |
| | | | HD NH NI NH NH | | ATTACACT |
| | | | NI NG NG NI HD | | |
| | | | NI HD NG | | |
| AJ877912.1 | 12 | 21 | HD HD NI NH NH | Plus | TCCAGGAA |
| | | | NI NI HD HD NH | | CCGAGGAT |
| | | | NI NH NH NI NG | | TACACT |
| | | | NG NI HD NI HD | | |
| | | | NG | | |
| AJ877912.1 | 28 | 17 | NI HD NI HD NG | Plus | TACACTCG |
| | | | HD NH HD NI NH | | CAGGAGCC |
| | | | NH NI NH HD HD | | TT |
| | | | NG NG | | |
| AJ877912.1 | 28 | 18 | NI HD NI HD NG | Plus | TACACTCG |
| | | | HD NH HD NI NH | | CAGGAGCC |
| | | | NH NI NH HD HD | | TTT |
| | | | NG NG NG | | |
| AJ877912.1 | 28 | 16 | NI HD NI HD NG | Plus | TACACTCG |
| | | | HD NH HD NI NH | | CAGGAGCC |
| | | | NH NI NH HD HD | | T |
| | | | NG | | |
| AJ877912.1 | 28 | 19 | NI HD NI HD NG | Plus | TACACTCG |
| | | | HD NH HD NI NH | | CAGGAGCC |
| | | | NH NI NH HD HD | | TTTT |
| | | | NG NG NG NG | | |
| AJ877912.1 | 28 | 26 | NI HD NI HD NG | Plus | TACACTCG |
| | | | HD NH HD NI NH | | CAGGAGCC |
| | | | NH NI NH HD HD | | TTTTAGCA |
| | | | NG NG NG NG NI | | AAT |
| | | | NH HD NI NI NI | | |
| | | | NG | | |
| AJ877912.1 | 33 | 21 | HD NH HD NI NH | Plus | TCGCAGGA |
| | | | NH NI NH HD HD | | GCCTTTTA |
| | | | NG NG NG NG NI | | GCAAAT |
| | | | NH HD NI NI NI | | |
| | | | NG | | |
| AJ877912.1 | 47 | 25 | NI NH HD NI NI | Plus | TAGCAAAT |
| | | | NI NG NH NH NH | | GGGCTTGG |
| | | | HD NG NG NH NH | | TCTAACAC |
| | | | NG HD NG NI NI | | CT |
| | | | HD NI HD HD NG | | |
| AJ877912.1 | 47 | 30 | NI NH HD NI NI | Plus | TAGCAAAT |
| | | | NI NG NH NH NH | | GGGCTTGG |
| | | | HD NG NG NH NH | | TCTAACAC |
| | | | NG HD NG NI NI | | CTCCGAT |
| | | | HD NI HD HD NG | | |
| | | | HD HD NH NI NG | | |
| AJ877912.1 | 60 | 17 | NH NH NG HD NG | Plus | TGGTCTAA |
| | | | NI NI HD NI HD | | CACCTCCG |
| | | | HD NG HD HD NH | | AT |
| | | | NI NG | | |
| AJ877912.1 | 82 | 18 | NH NH NI NI HD | Plus | TGGAACAG |
| | | | NI NH HD HD NH | | CCGCAATC |
| | | | HD NI NI NG HD | | ATT |
| | | | NI NG NG | | |

Kopischke S, Schüβler E, Althoff F, Zachgo S. Plant Methods. 2017 Mar. 29; 13:20;
Zhang K, Raboanatahiry N, Zhu B, Li M. Front Plant Sci. 2017 Feb. 14; 8:177;
Jung J H, Altpeter F. Plant Mol Biol. 2016 September; 92(1-2):131-42;
Li T, Liu B, Chen C Y, Yang B. J Genet Genomics. 2016 May 20; 43(5):297-305; Blanvillain-Baufumé S, Reschke M, Solé M, Auguy F, Doucoure H, Szurek B, Meynard D, Portefaix M, Cunnac S, Guiderdoni E, Boch J, Koebnik R. Plant Biotechnol J. 2017 March; 15(3):306-317).

According to a specific embodiment, the nucleic acid construct further comprises a nucleic acid sequence encoding an endonuclease of a DNA editing agent (e.g., Cas9 or the endonucleases described above).

According to another specific embodiment, the endonuclease and the sgRNA are encoded from different constructs whereby each is operably linked to a cis-acting regulatory element active in plant cells (e.g., promoter).

In a particular embodiment of some embodiments of the invention the regulatory sequence is a plant-expressible promoter.

Constructs useful in the methods according to some embodiments may be constructed using recombinant DNA technology well known to persons skilled in the art. Such constructs may be commercially available, suitable for transforming into plants and suitable for expression of the gene of interest in the transformed cells.

As used herein the phrase “plant-expressible” refers to a promoter sequence, including any additional regulatory elements added thereto or contained therein, is at least capable of inducing, conferring, activating or enhancing expression in a plant cell, tissue or organ, preferably a monocotyledonous or dicotyledonous plant cell, tissue, or organ. Examples of promoters useful for the methods of some embodiments of the invention include, but are not limited to, Actin, CANV 35S, CaMV19S, GOS2. Promoters which are active in various tissues, or developmental stages can also be used.

Nucleic acid sequences of the polypeptides of some embodiments of the invention may be optimized for plant expression. Examples of such sequence modifications include, but are not limited to, an altered G/C content to more closely approach that typically found in the plant species of interest, and the removal of codons atypically found in the plant species commonly referred to as codon optimization.

Plant cells may be transformed stably or transiently with the nucleic acid constructs of some embodiments of the invention. In stable transformation, the nucleic acid molecule of some embodiments of the invention is integrated into the plant genome and as such it represents a stable and inherited trait. In transient transformation, the nucleic acid molecule is expressed by the cell transformed but it is not integrated into the genome and as such it represents a transient CRISPR-Cas9 system.

According to a specific embodiment, the plant is transiently transfected with a DNA editing agent.

According to a specific embodiment, promoters in the nucleic acid construct comprise a Pol3 promoter. Examples of Pol3 promoters include, but are not limited to, AtU6-29, AtU626, AtU3B, AtU3d, TaU6.

According to a specific embodiment, promoters in the nucleic acid construct comprise a Pol2 promoter. Examples of Pol2 promoters include, but are not limited to, CaMV 35S, CaMV 19S, ubiquitin, CVMV.

According to a specific embodiment, promoters in the nucleic acid construct comprise a 35S promoter.

According to a specific embodiment, promoters in the nucleic acid construct comprise a U6 promoter.

According to a specific embodiment, promoters in the nucleic acid construct comprise a Pol 3 (e.g., U6) promoter operatively linked to the nucleic acid agent encoding at least one gRNA and/or a Pol2 (e.g., CaMV35S) promoter operatively linked to the nucleic acid sequence encoding the genome editing agent or the nucleic acid sequence encoding the fluorescent reporter (as described in a specific embodiment below).

According to a specific embodiment, the construct is useful for transient expression (Helens et al., 2005, Plant Methods 1:13). Methods of transient transformation are further described herein.

According to a specific embodiment, the nucleic acid sequences comprised in the construct are devoid of sequences which are homologous to the plant cell's genome so as to avoid integration to the plant genome.

In certain embodiments, the nucleic acid construct is a non-integrating construct, preferably where the nucleic acid sequence encoding the fluorescent reporter is also non-integrating. As used herein, “non-integrating” refers to a construct or sequence that is not affirmatively designed to facilitate integration of the construct or sequence into the genome of the plant of interest. For example, a functional T-DNA vector system for Agrobacterium-mediated genetic transformation is not a non-integrating vector system as the system is affirmatively designed to integrate into the plant genome. Similarly, a fluorescent reporter gene sequence or selectable marker sequence that has flanking sequences that are homologous to the genome of the plant of interest to facilitate homologous recombination of the fluorescent reporter gene sequence or selectable marker sequence into the genome of the plant of interest would not be a non-integrating fluorescent reporter gene sequence or selectable marker sequence.

Various cloning kits can be used according to the teachings of some embodiments of the invention.

According to a specific embodiment the nucleic acid construct is a binary vector. Examples for binary vectors are pBIN19, pBI01, pBinAR, pGPTV, pCAMBIA, pBIB-HYG, pBecks, pGreen or pPZP (Hajukiewicz, P. et al., Plant Mol. Biol. 25, 989 (1994), and Hellens et al, Trends in Plant Science 5, 446 (2000)).

Examples of other vectors to be used in other methods of DNA delivery (e.g. transfection, electroporation, bombardment, viral inoculation) are: pGE-sgRNA (Zhang et al. Nat. Comms. 2016 7:12697), pJIT163-Ubi-Cas9 (Wang et al. Nat. Biotechnol 2004 32, 947-951), pICH47742::2x35S-5′UTR-hCas9(STOP)-NOST (Belhan et al. Plant Methods 2013 11; 9(1):39).

Embodiments described herein also relate to a method of selecting cells comprising a genome editing event, the method comprising:

(a) transforming cells of a coffee plant with a nucleic acid construct comprising the genome editing agent (as described above) and a fluorescent reporter;

(b) selecting transformed cells exhibiting fluorescence emitted by the fluorescent reporter using flow cytometry or imaging;

(c) culturing the transformed cells comprising the genome editing event by the DNA editing agent for a time sufficient to lose expression of the DNA editing agent so as to obtain cells which comprise a genome editing event generated by the DNA editing agent but lack DNA encoding the DNA editing agent; and

According to some embodiments, the method further comprises validating in the transformed cells, loss of expression of the fluorescent reporter following step (c).

According to some embodiments, the method further comprises validating in the transformed cells loss, of expression/occurrence of the DNA editing agent following step (c).

A non-limiting embodiment of the method is described in the Flowchart of FIG. 1.

According to a specific embodiment, the plant is a plant cell e.g., plant cell in an embryonic cell suspension.

According to a specific embodiment, the plant cell is a protoplast.

The protoplasts are derived from any plant tissue e.g., roots, leaves, embryonic cell suspension, calli or seedling tissue.

There are a number of methods of introducing DNA into plant cells e.g., using protoplasts and the skilled artisan will know which to select.

The delivery of nucleic acids may be introduced into a plant cell in embodiments of the invention by any method known to those of skill in the art, including, for example and without limitation: by transformation of protoplasts (See, e.g., U.S. Pat. No. 5,508,184); by desiccation/inhibition-mediated DNA uptake (See, e.g., Potrykus et al. (1985) Mol. Gen. Genet. 199:183-8); by electroporation (See, e.g., U.S. Pat. No. 5,384,253); by agitation with silicon carbide fibers (See, e.g., U.S. Pat. Nos. 5,302,523 and 5,464,765); by Agrobacterium-mediated transformation (See, e.g., U.S. Pat. Nos. 5,563,055, 5,591,616, 5,693,512, 5,824,877, 5,981,840, and 6,384,301); by acceleration of DNA-coated particles (See, e.g., U.S. Pat. Nos. 5,015,580, 5,550,318, 5,538,880, 6,160,208, 6,399,861, and 6,403,865) and by Nanoparticles, nanocarriers and cell penetrating peptides (WO201126644A2; WO2009046384A1; WO2008148223A1) in the methods to deliver DNA, RNA, Peptides and/or proteins or combinations of nucleic acids and peptides into plant cells.

Other methods of transfection include the use of transfection reagents (e.g. Lipofectin, ThermoFisher), dendrimers (Kukowska-Latallo, J. F. et al., 1996, Proc. Natl. Acad. Sci. USA93, 4897-902), cell penetrating peptides (Mie et al., 2005, Internalisation of cell-penetrating peptides into tobacco protoplasts, Biochimica et Biophysica Acta 1669(2):101-7) or polyamines (Zhang and Vinogradov, 2010, Short biodegradable polyamines for gene delivery and transfection of brain capillary endothelial cells, J Control Release, 143(3):359-366).

According to a specific embodiment, the introduction of DNA into plant cells (e.g., protoplasts) is effected by electroporation.

According to a specific embodiment, the introduction of DNA into plant cells (e.g., protoplasts) is effected by bombardment/biolistics.

According to a specific embodiment, for introducing DNA into protoplasts the method comprises polyethylene glycol (PEG)-mediated DNA uptake. For further details see Karesch et al. (1991) Plant Cell Rep. 9:575-578; Mathur et al. (1995) Plant Cell Rep. 14:221-226; Negrutiu et al. (1987) Plant Cell Mol. Biol. 8:363-373. Protoplasts are then cultured under conditions that allowed them to grow cell walls, start dividing to form a callus, develop shoots and roots, and regenerate whole plants.

Transient transformation can also be effected by viral infection using modified plant viruses.

Viruses that have been shown to be useful for the transformation of plant hosts include CaMV, TMV, TRV and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

Construction of plant RNA viruses for the introduction and expression of non-viral exogenous nucleic acid sequences in plants is demonstrated by the above references as well as by Dawson, W. O. et al. Virology (1989) 172:285-292; Takamatsu et al. EMBO J. (1987) 6:307-311; French et al. Science (1986) 231:1294-1297; and Takamatsu et al. FEBS Letters (1990) 269:73-76.

When the virus is a DNA virus, suitable modifications can be made to the virus itself. Alternatively, the virus DNA can first be cloned into a bacterial plasmid for ease of constructing the desired viral vector with the foreign DNA. The virus DNA can then be excised from the plasmid. If the virus is a DNA virus, a bacterial origin of replication can be attached to the viral DNA, which is then replicated by the bacteria. Transcription and translation of this DNA will produce the coat protein which will encapsidate the viral DNA. If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

Construction of plant RNA viruses for the introduction and expression in plants of non-viral exogenous nucleic acid sequences such as those included in the construct of some embodiments of the invention is demonstrated by the above references as well as in U.S. Pat. No. 5,316,931.

In one embodiment, a plant viral nucleic acid is provided in which the native coat protein coding sequence has been deleted from a viral nucleic acid, a non-native plant viral coat protein coding sequence and a non-native promoter, preferably the subgenomic promoter of the non-native coat protein coding sequence, capable of expression in the plant host, packaging of the recombinant plant viral nucleic acid, and ensuring a systemic infection of the host by the recombinant plant viral nucleic acid, has been inserted. Alternatively, the coat protein gene may be inactivated by insertion of the non-native nucleic acid sequence within it, such that a protein is produced. The recombinant plant viral nucleic acid may contain one or more additional non-native subgenomic promoters. Each non-native subgenomic promoter is capable of transcribing or expressing adjacent genes or nucleic acid sequences in the plant host and incapable of recombination with each other and with native subgenomic promoters. Non-native (foreign) nucleic acid sequences may be inserted adjacent the native plant viral subgenomic promoter or the native and a non-native plant viral subgenomic promoters if more than one nucleic acid sequence is included. The non-native nucleic acid sequences are transcribed or expressed in the host plant under control of the subgenomic promoter to produce the desired products.

In a second embodiment, a recombinant plant viral nucleic acid is provided as in the first embodiment except that the native coat protein coding sequence is placed adjacent one of the non-native coat protein subgenomic promoters instead of a non-native coat protein coding sequence.

In a third embodiment, a recombinant plant viral nucleic acid is provided in which the native coat protein gene is adjacent its subgenomic promoter and one or more non-native subgenomic promoters have been inserted into the viral nucleic acid. The inserted non-native subgenomic promoters are capable of transcribing or expressing adjacent genes in a plant host and are incapable of recombination with each other and with native subgenomic promoters. Non-native nucleic acid sequences may be inserted adjacent the non-native subgenomic plant viral promoters such that said sequences are transcribed or expressed in the host plant under control of the subgenomic promoters to produce the desired product.

In a fourth embodiment, a recombinant plant viral nucleic acid is provided as in the third embodiment except that the native coat protein coding sequence is replaced by a non-native coat protein coding sequence.

The viral vectors are encapsidated by the coat proteins encoded by the recombinant plant viral nucleic acid to produce a recombinant plant virus. The recombinant plant viral nucleic acid or recombinant plant virus is used to infect appropriate host plants. The recombinant plant viral nucleic acid is capable of replication in the host, systemic spread in the host, and transcription or expression of foreign gene(s) (isolated nucleic acid) in the host to produce the desired protein.

Regardless of the transformation/infection method employed, the present teachings further relate to any cell e.g., a plant cell (e.g., protoplast) or a bacterial cell comprising the nucleic acid construct(s) as described herein.

Following transformation, cells are subjected to flow cytometry to select transformed cells exhibiting fluorescence emitted by the fluorescent reporter (i.e., fluorescent protein”).

As used herein, “a fluorescent protein” refers to a polypeptide that emits fluorescence and is typically detectable by flow cytometry or imaging, therefore can be used as a basis for selection of cells expressing such a protein.

Examples of fluorescent proteins that can be used as reporters are the Green Fluorescent Protein (GFP), the Blue Fluorescent Protein (BFP) and the red fluorescent protein dsRed. A non-limiting list of fluorescent or other reporters includes proteins detectable by luminescence (e.g. luciferase) or colorimetric assay (e.g. GUS). According to a specific embodiment, the fluorescent reporter is DsRed or GFP.

This analysis is typically effected within 24-72 hours e.g., 48-72, 24-28 hours, following transformation. To ensure transient expression, no antibiotic selection is employed e.g., antibiotics for a selection marker. The culture may still comprise antibiotics but not to a selection marker.

Flow cytometry of plant cells is typically performed by Fluorescence Activated Cell Sorting (FACS). Fluorescence activated cell sorting (FACS) is a well-known method for separating particles, including cells, based on the fluorescent properties of the particles (see, e.g., Kamarch, 1987, Methods Enzymol, 151:150-165).

For instance, FACS of GFP-positive cells makes use of the visualization of the green versus the red emission spectra of protoplasts excited by a 488 nm laser. GFP-positive protoplasts can be distinguished by their increased ratio of green to red emission.

Following is a non-binding protocol adapted from Bastiaan et al. J Vis Exp. 2010; (36): 1673, which is hereby incorporated by reference. FACS apparati are commercially available e.g., FACSMelody (BD), FACSAria (BD).

A flow stream is set up with a 100 μm nozzle and a 20 psi sheath pressure. The cell density and sample injection speed can be adjusted to the particular experiment based on whether a best possible yield or fastest achievable speed is desired, e.g., up to 10,000,000 cells/ml. The sample is agitated on the FACS to prevent sedimentation of the protoplasts. If clogging of the FACS is an issue, there are three possible troubleshooting steps: 1. Perform a sample-line backflush. 2. Dilute protoplast suspension to reduce the density. 3. Clean up the protoplast solution by repeating the filtration step after centrifugation and resuspension. The apparatus is prepared to measure forward scatter (FSC), side scatter (SSC) and emission at 530/30 nm for GFP and 610/20 nm for red spectrum auto-fluorescence (RSA) after excitation by a 488 nm laser. These are in essence the only parameters used to isolate GFP-positive protoplasts. The voltage settings can be used: FSC—60V, SSC 250V, GFP 350V and RSA 335V. Note that the optimal voltage settings will be different for every FACS and will even need to be adjusted throughout the lifetime of the cell sorter.

The process is started by setting up a dotplot for forward scatter versus side scatter. The voltage settings are applied so that the measured events are centered in the plot. Next, a dot plot is created of green versus red fluorescence signals. The voltage settings are applied so that the measured events yield a centered diagonal population in the plot when looking at a wild-type (non-GFP) protoplast suspension. A protoplast suspension derived from a GFP marker line will produce a clear population of green fluorescent events never seen in wild-type samples. Compensation constraints are set to adjust for spectral overlap between GFP and RSA. Proper compensation constraint settings will allow for better separation of the GFP-positive protoplasts from the non-GFP protoplasts and debris. The constraints used here are as follows: RSA, minus 17.91% GFP. A gate is set to identify GFP-positive events, a negative control of non-GFP protoplasts should be used to aid in defining the gate boundaries. A forward scatter cutoff is implemented in order to leave small debris out of the analysis. The GFP-positive events are visualized in the FSC vs. SSC plot to help determine the placement of the cutoff. E.g., cutoff is set at 5,000. Note that the FACS will count debris as sort events and a sample with high levels of debris may have a different percent GFP positive events than expected. Ibis is not necessarily a problem. However, the more debris in the sample, the longer the sort will take. Depending on the experiment and the abundance of the cell type to be analyzed, the FACS precision mode is set either for optimal yield or optimal purity of the sorted cells.

Following FACS sorting, positively selected pools of transformed plant cells, (e.g., protoplasts) displaying the fluorescent marker are collected and an aliquot can be used for testing the DNA editing event (optional step, see FIG. 1). Alternatively (or following optional validating) the clones are cultivated in the absence of selection (e.g., antibiotics for a selection marker) until they develop into colonies i.e., clones (at least 28 days) and micro-calli. Following at least 60-100 days in culture (e.g., at least 70 days, at least 80 days), a portion of the cells of the calli are analyzed (validated) for: the DNA editing event and the presence of the DNA editing agent, namely, loss of DNA sequences encoding for the DNA editing agent, pointing to the transient nature of the method.

Thus, clones are validated for the presence of a DNA editing event also referred to herein as “mutation” or “edit”, dependent on the type of editing sought e.g., insertion, deletion, insertion-deletion (Indel), inversion, substitution and combinations thereof.

According to a specific embodiment, the genome editing event comprises a deletion, a single base pair substitution, or an insertion of genetic material from a second plant that could otherwise be introduced into the plant of interest by traditional breeding.

According to a specific embodiment, the genome editing event does not comprise an introduction of foreign DNA into a genome of the plant of interest that could not be introduced through traditional breeding.

Methods for detecting sequence alteration are well known in the art and include, but not limited to, DNA sequencing (e.g., next generation sequencing), electrophoresis, an enzyme-based mismatch detection assay and a hybridization assay such as PCR, RT-PCR, RNase protection, in-situ hybridization, primer extension, Southern blot, Northern Blot and dot blot analysis. Various methods used for detection of single nucleotide polymorphisms (SNPs) can also be used, such as PCR based T7 endonuclease, Hetroduplex and Sanger sequencing.

Another method of validating the presence of a DNA editing event e.g., Indels comprises a mismatch cleavage assay that makes use of a structure selective enzyme (e,g,m endonuclease) that recognizes and cleaves mismatched DNA.

The mismatch cleavage assay is a simple and cost-effective method for the detection of indels and is therefore the typical procedure to detect mutations induced by genome editing. The assay uses enzymes that cleave heteroduplex DNA at mismatches and extrahelical loops formed by multiple nucleotides, yielding two or more smaller fragments. A PCR product of ˜300-1000 bp is generated with the predicted nuclease cleavage site off-center so that the resulting fragments are dissimilar in size and can easily be resolved by conventional gel electrophoresis or high-performance liquid chromatography (HPLC). End-labeled digestion products can also be analyzed by automated gel or capillary electrophoresis. The frequency of indels at the locus can be estimated by measuring the integrated intensities of the PCR amplicon and cleaved DNA bands. The digestion step takes 15-60 min, and when the DNA preparation and PCR steps are added the entire assays can be completed in <3 h.

Two alternative enzymes are typically used in this assay. T7 endonuclease 1 (T7E1) is a resolvase that recognizes and cleaves imperfectly matched DNA at the first, second or third phosphodiester bond upstream of the mismatch. The sensitivity of a T7E1-based assay is 0.5-5%. In contrast, Surveyor™ nuclease (Transgenomic Inc., Omaha, Nebr., USA) is a member of the CEL family of mismatch-specific nucleases derived from celery. It recognizes and cleaves mismatches due to the presence of single nucleotide polymorphisms (SNPs) or small indels, cleaving both DNA strands downstream of the mismatch. It can detect indels of up to 12 nt and is sensitive to mutations present at frequencies as low as ˜3%, i.e. 1 in 32 copies.

Yet another method of validating the presence of an editing even comprises the high-resolution melting analysis.

High-resolution melting analysis (HRMA) involves the amplification of a DNA sequence spanning the genomic target (90-200 bp) by real-time PCR with the incorporation of a fluorescent dye, followed by melt curve analysis of the amplicons. HRMA is based on the loss of fluorescence when intercalating dyes are released from double-stranded DNA during thermal denaturation. It records the temperature-dependent denaturation profile of amplicons and detects whether the melting process involves one or more molecular species.

Yet another method is the heteroduplex mobility assay. Mutations can also be detected by analyzing re-hybridized PCR fragments directly by native polyacrylamide gel electrophoresis (PAGE). This method takes advantage of the differential migration of heteroduplex and homoduplex DNA in polyacrylamide gels. The angle between matched and mismatched DNA strands caused by an indel means that heteroduplex DNA migrates at a significantly slower rate than homoduplex DNA under native conditions, and they can easily be distinguished based on their mobility. Fragments of 140-170 bp can be separated in a 15% polyacrylamide gel. The sensitivity of such assays can approach 0.5% under optimal conditions, which is similar to T7E1 (. After reannealing the PCR products, the electrophoresis component of the assay takes ˜2 h.

Other methods of validating the presence of editing events are described in length in Zischewski 2017 Biotechnol. Advances 1(1):95-104.

It will be appreciated that positive clones can be homozygous or heterozygous for the DNA editing event. The skilled artisan will select the clone for further culturing/regeneration according to the intended use.

Clones exhibiting the presence of a DNA editing event as desired are further analyzed for the presence of the DNA editing agent. Namely, loss of DNA sequences encoding for the DNA editing agent, pointing to the transient nature of the method.

This can be done by analyzing the expression of the DNA editing agent (e.g., at the mRNA, protein) e.g., by fluorescent detection of GFP or q-PCR.

Alternatively or additionally, the cells are analyzed for the presence of the nucleic acid construct as described herein or portions thereof e.g., nucleic acid sequence encoding the reporter polypeptide or the DNA editing agent.

Clones showing no DNA encoding the fluorescent reporter or DNA editing agent (e.g., as affirmed by fluorescent microscopy, q-PCR and or any other method such as Southern blot, PCR, sequencing) yet comprising the DNA editing event(s) [mutation(s)] as desired are isolated for further processing.

These clones can therefore be stored (e.g., cryopreserved).

Alternatively, cells (e.g., protoplasts) may be regenerated into whole plants first by growing into a group of plant cells that develops into a callus and then by regeneration of shoots (caulogenesis) from the callus using plant tissue culture methods. Growth of protoplasts into callus and regeneration of shoots requires the proper balance of plant growth regulators in the tissue culture medium that must be customized for each species of plant

Protoplasts may also be used for plant breeding, using a technique called protoplast fusion. Protoplasts from different species are induced to fuse by using an electric field or a solution of polyethylene glycol. This technique may be used to generate somatic hybrids in tissue culture.

Methods of protoplast regeneration are well known in the art. Several factors affect the isolation, culture, and regeneration of protoplasts, namely the genotype, the donor tissue and its pre-treatment, the enzyme treatment for protoplast isolation, the method of protoplast culture, the culture, the culture medium, and the physical environment. For a thorough review see Maheshwari et al. 1986 Differentiation of Protoplasts and of Transformed Plant Cells: 3-36. Springer-Verlag, Berlin.

The regenerated plants can be subjected to further breeding and selection as the skilled artisan sees fit.

The phenotype of the final lines, plants or intermediate breeding products can be analyzed such as by determining the sequence of the α-D-galactosidase gene, expression thereof in the mRNA or protein level, activity of the protein and/or analyzing the properties of the coffee been (solubility).

For example, plant material is ground in liquid nitrogen and extracted in ice cold enzyme extraction buffer (glycerol 10% v/v, sodium metabisulfite 10 mM, EDTA 5 mM, MOPS (NaOH) 40 mM, pH 6.5) at an approximate ratio of 20 mg per 100 μl. The mixture is stirred on ice for 20 min, subjected to centrifugation (12,000 g×30 min), aliquoted and stored at −85° C. until use. α-D-galactosidase activity is detected spectrophotometrically with the substrate p-nitrophenyl-α-D-galactopyranoside (pNGP).

The reaction mixture contains 200 μl pNGP 100 mM in McIlvain's buffer (citric acid 100 mM-Na₂HPO₄ 200 mM pH 6.5) up to final volume of 1 ml with enzyme extract. The reaction is maintained at 26° C. and started with the addition of enzyme and is stopped by addition of 4 volumes of stop solution (Na₂CO₃—NaHCO₃100 mM pH 10.2). Absorption is read at 405 nm. Evolution of nitrophenyl is calculated using molar extinction coefficient ε=18300 (specific for pH 10.2) and converted to mmol min⁻¹ mg protein⁻¹. Total protein is measured in samples extracted in aqueous buffers by the method of Bradford (Anal. Biochem., 72 (1976), 248-254). For the expression of activity, each sample is extracted and aliquoted, and assays are performed in triplicate, the results being expressed as averages.

As is illustrated herein and in the Examples section which follows. The present inventors were able to transform coffee while avoiding stable transgenesis.

Hence the present methodology allows genome editing without integration of a selectable or screenable reporter.

Thus, embodiments of the invention further relate to non-transgenic plants, non-transgenic plant cells and processed product of plants comprising the gene editing event(s) generated according to the present teachings,

Thus, the present teachings also relate to parts of the plants as described herein or processed products thereof.

According to some embodiments there is provided a method of producing soluble coffee, the method comprising subjecting beans of the coffee as described herein to extraction, dehydration and optionally roasting.

According to a specific embodiment, processed products of the plants comprise DNA including the mutated α-D-galactosidase gene that imparts the increased solubility.

Processed coffee compositions of some embodiments can be in the form of a coffee powder to be extracted or brewed or a soluble coffee powder. Thus, it can be coarse-ground coffee, filter coffee or instant coffee. On the other hand, the coffee composition of the invention can also comprise whole roasted coffee beans. Further embodiments of the invention relate to a coffee beverage comprising the coffee composition and water. Such a coffee beverage can be prepared with methods known to a person skilled in the art, such as by extracting with water, brewing in water or soaking the coffee composition of the invention in water. The coffee beverage of the invention can also comprise other substances, such as natural or artificial flavouring substances, milk products, alcohol, foaming agents, natural or artificial sweetening agents, and the like.

It is expected that during the life of a patent maturing from this application many relevant DNA editing agents will be developed and the scope of the term DNA editing agent is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals in between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. For example, a given SEQ ID NO: is expressed in a DNA sequence format (e.g., reciting T for thymine), but it can refer to either a DNA sequence that corresponds to a given nucleic acid sequence, or the RNA sequence of an RNA molecule nucleic acid sequence. Similarly, though some sequences are expressed in a RNA sequence format (e.g., reciting U for uracil), depending on the actual type of molecule being described, it can refer to either the sequence of a RNA molecule comprising a dsRNA, or the sequence of a DNA molecule that corresponds to the RNA sequence shown. In any event, both DNA and RNA molecules having the sequences disclosed with any substitutes are envisioned.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. For example, a given SEQ ID NO: is expressed in a DNA sequence format (e.g., reciting T for thymine), but it can refer to either a DNA sequence or the RNA sequence of an RNA molecule nucleic acid sequence. Similarly, though some sequences are expressed in a RNA sequence format (e.g., reciting U for uracil), depending on the actual type of molecule being described, it can refer to either the sequence of a RNA molecule comprising a dsRNA, or the sequence of a DNA molecule that corresponds to the RNA sequence shown. In any event, both DNA and RNA molecules having the sequences disclosed with any substitutes are envisioned.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Materials and Methods

Protoplast Isolation from Embryonic Callus

Embryonic calli are obtained as previously described [Etienne, H., Somatic embryogenesis protocol: coffee (Coffea arabica L. and C. canephora P.), in Protocol for somatic embryogenesis in woody plants. 2005, Springer. p. 167-1795]. Briefly, young leaves of coffee are surface sterilized, cut into 1 cm² pieces and placed on half strength semi solid MS medium supplemented with 2.26 μM 2,4-dichlorophenoxyacetic acid (2,4-D), 4.92 μM indole-3-butyric acid (IBA) and 9.84 μM isopentenyladenine (iP) for one month. Explants are then transferred to half strength semisolid MS medium containing 4.52 μM 2,4-D and 17.76 μM 6-benzylaminopurine (6-BAP) for 6 to 8 months until regeneration of embryogenic calli. Embryogenic calli are maintained on MS media supplemented with 5 μM 6-BAP.

Cell suspension cultures are generated from embryogenic calli as previously described [Acuna, J. R. and M. de Pena, Plant regeneration from protoplasts of embryogenic cell suspensions of Coffea arabica L. cv. caturra. Plant Cell Reports, 1991. 10(6): p. 345-348]. Embryogenic calli (30 g/l) are placed in liquid MS medium supplemented with 13.32 μM 6-BAP. Flasks are placed in a shaking incubator (110 rpm) at 28° C. The cell suspension is subcultured/passaged every two to four weeks until fully established. Cell suspension cultures are maintained in liquid MS medium with 4.44 μM 6-BAP. Protoplasts are generated as previously described [Acuna, J. R. and M. de Pena, Plant regeneration from protoplasts of embryogenic cell suspensions of Coffea arabica L. cv. caturra. Plant Cell Reports, 1991. 10(6): p. 345-348; Yamada, Y., Z. Q. Yang, and D. T. Tang, Plant regeneration from protoplast-derived callus of rice (Oryza sativa L.). Plant Cell Rep, 1986. 5(2): p. 85-8] and for review see [Davey, M. R., et al., Plant protoplasts: status and biotechnological perspectives. Biotechnol Adv, 2005. 23(2): p. 131-71]. In brief approximately 0.5 grams of cells are collected from 5-6 day old suspension cultures and incubated with gentle agitation in a culture which contains Cellulase Onozuka R-10 (2%), Pectolyase Y-23 (0.2%) and Driselase (0.2%) dissolved in protoplast culture medium (mannitol 0.4M, NaCl, 154 mM; CaCl₂), 125 mM; KCl, 5 mM; MES-K, 2 mM) for 4-6 hours. Protoplasts are washed, purified by filtration (70 microns) and subsequent flotation on 40% Percoll (sigma) or a cushion of 20% sucroseCell density is determined using a haemocytometer and the viability of protoplasts is determined by staining with 0.01% (w/v) fluorescein diacetate (FDA) and observation under a fluorescent microscope.

Target Gene

The target gene in cultivar C. canephora is alpha-D-Galactosidase >chr4 chr4:20969056..20978218 (+strand) class=gene length=9163 (SEQ ID NO: 4 Cc04_g14280).

sgRNAs Design

sgRNAs are designed using the publically available sgRNA designer, from Park, J., S. Bae, and J.-S. Kim, Cas-Designer: a web-based tool for choice of CRISPR-Cas9 target sites. Bioinformatics, 2015. 31(24): p. 4014-4016. Two sgRNAs are designed for α-d-galactosidase gene to increase the chances of a DSBs which could result in the loss of function of the target gene.

Cc04_g14280
(SEQ ID NO: 13)
GGTGAAGTCTCCAGGAACCG;
(SEQ ID NO: 14)
GCTTGGTCTAACACCTCCGA;

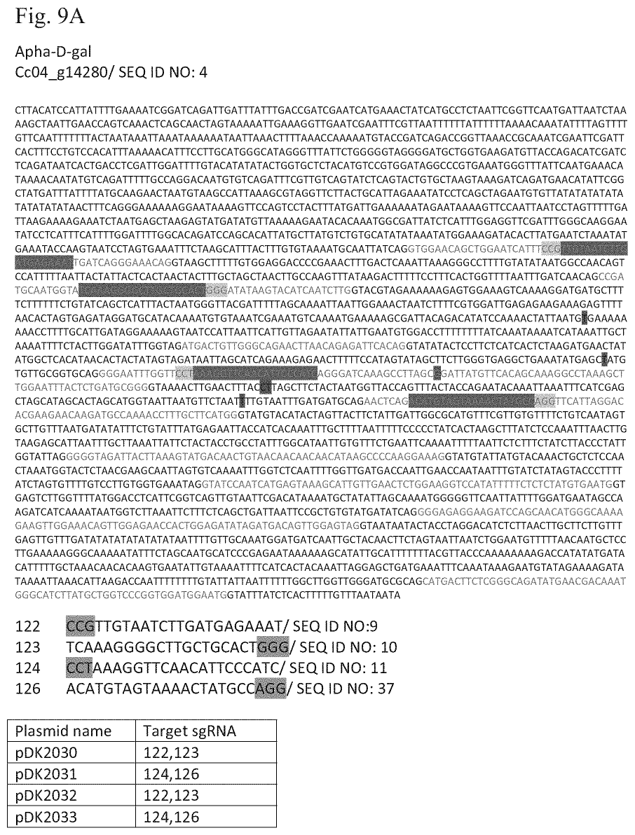

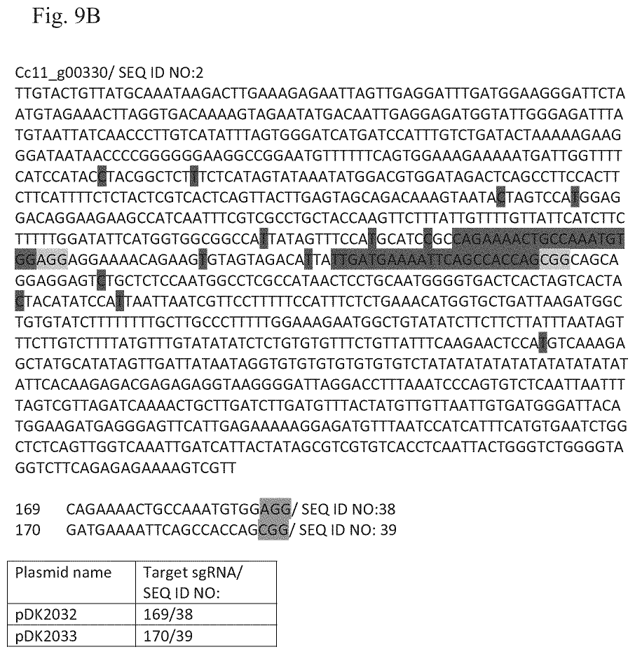

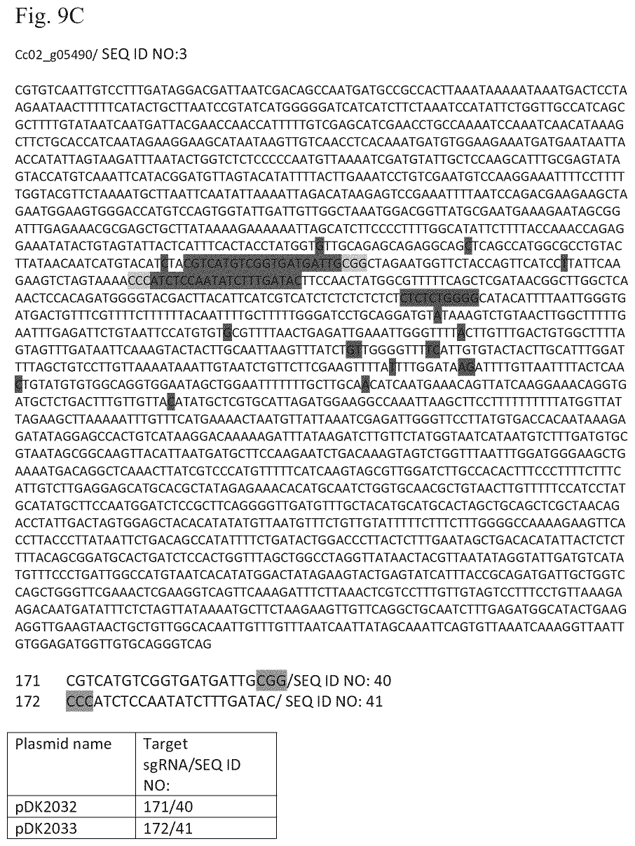

See also sgRNAs in FIG. 9A-C for Cc04_g14280, Cc11_g00330 and Cc02_g05490.

sgRNA Cloning

The transfection plasmid utilized was composed of 4 modules comprising of 1, eGFP driven by the CaMV35s promoter terminated by a G7 temination sequence; 2, Cas9 (human codon optimised) driven by the CaMV35s promoter terminated by Mas termination sequence; 3, AtU6 promoter driving sgRNA for guide 1; 4 AtU6 promoter driving sgRNA for guide 2. A binary vector can be used such as pCAMBIA or pRI-201-AN DNA.

Polyethylene glycol (PEG)-mediated plasmid transfection. PEG-transfection of coffee and banana protoplasts was effected using a modified version of the strategy reported by Wang et al., (2015) [Wang, H., et al., An efficient PEG-mediated transient gene expression system in grape protoplasts and its application in subcellular localization studies of flavonoids biosynthesis enzymes. Scientia Horticulturae, 2015. 191: p. 82-89]. Protoplasts were resuspended to a density of 2-5×10⁶ protoplasts/ml in MMg solution. 100-200 μl of protoplast suspension was added to a tube containing the plasmid. The plasmid:protoplast ratio greatly affects transformation efficiency therefore a range of plasmid concentrations in protoplast suspension, 5-300 μg/μl, were assayed. PEG solution (100-200 μl) was added to the mixture and incubated at 23° C. for various lengths of time ranging from 10-60 minutes. PEG4000 concentration was optimized, a range of 20-80% PEG4000 in 200-400 mM mannitol, 100-500 mM CaCl₂) solution was assayed. The protoplasts were then washed in W5 and centrifugated at 80 g for 3 min, prior resuspension in 1 ml W5 and incubated in the dark at 23° C. After incubation for 24-72 h fluorescence was detected by microscopy.

Electroporation

A plasmid containing Pol2-driven GFP/RFP, Pol2-driven-NLS-Cas9 and Pol3-driven sgRNA targeting the relevant genes was introduced to the cells using electroporation (BIORAD-GenePulserII; Miao and Jian 2007 Nature Protocols 2(10): 2348-2353. 500 μl of protoplasts were transferred into electroporation cuvettes and mixed with 100 μl of plasmid (10-40 μg DNA). Protoplasts were electroporated at 130 V and 1,000 F and incubated at room temperature for 30 minutes. 1 ml of protoplast culture medium was added to each cuvette and the protoplast suspension was poured into a small petri dish. After incubation for 24-48 h fluorescence was detected by microscopy.

FACS Sorting of Fluorescent Protein-Expressing Cells

48 hrs after plasmid/RNA delivery, cells were collected and sorted for fluorescent protein expression using a flow cytometer in order to enrich for GFP/Editing agent expressing cells [Chiang, T. W., et al., CRISPR-Cas9(D10A) nickase-based genotypic and phenotypic screening to enhance genome editing. Sci Rep, 2016. 6: p. 24356]. This enrichment step allows bypassing marker (e.g., antibiotic) selection and collecting only cells transiently expressing the fluorescent protein, Cas9 and the sgRNA. These cells can be further tested for editing of the target gene by non-homologues end joining (NHEJ) and loss of the corresponding gene expression.

Colony Formation

The fluorescent protein positive cells were partly sampled and used for DNA extraction and genome editing (GE) testing and partly plated at high dilution in liquid medium to allow colony formation for 28-35 days. Colonies were picked, grown and split into two aliquots. One aliquot was used for DNA extraction and genome editing (GE) testing and CRISPR DNA-free testing (see below), while the others were kept in culture until their status was verified. Only the ones clearly showing to be GE and CRISPR DNA-free were selected forward.

After 20 days in the dark (from splitting for GE analysis, i.e., 60 days, hence 80 days in total), the colonies were transferred to the same medium but with reduced glucose (0.46 M) and 0.4% agarose and incubated at a low light intensity. After six weeks agarose was cut into slices and placed on protoplast culture medium with 0.31 M glucose and 0.2% gelrite. After one month, protocolonies (or calli) were subcultured into regeneration media (half strength MS+B5 vitamins, 20 g/l sucrose). Regenerated plantlets were placed on solidified media (0.8% agar) at a low light intensity at 28° C. After 2 months plantlets were transferred to soil and placed in a glasshouse at 80-100% humidity.

Screen for Gene Modification and Absence of CRISPR System DNA

From each colony DNA was extracted from an aliquot of GFP-sorted protoplasts (optional step) and from protoplasts-derived colonies and a PCR reaction was performed with primers flanking the targeted gene. Measures are taken to sample the colony as positive colonies will be used to regenerate the plant. A control reaction from protoplasts subjected to the same method but without Cas9-sgRNA is included and considered as wild type (WT). The PCR products were then separated on an agarose gel to detect any changes in the product size compared to the WT. The PCR reaction products that vary from the WT products were cloned into pBLUNT or PCR-TOPO (Invitrogen). Alternatively, sequencing was used to verify the editing event. The resulting colonies were picked, plasmids were isolated and sequenced to determine the nature of the mutations. Clones (colonies or calli) harbouring mutations that were predicted to result in domain-alteration or complete loss of the corresponding protein were chosen for whole genome sequencing in order to validate that they were free from the CRISPR system DNA/RNA and to detect the mutations at the genomic DNA level.

Positive clones exhibiting the desired GE were first tested for GFP expression via microscopy analysis (compared to WT). Next, GFP-negative plants were tested for the presence of the Cas9 cassette by PCR using primers specific (or next generation sequencing, NGS) for the Cas9 sequence or any other sequence of the expression cassette. Other regions of the construct can also be tested to ensure that nothing of the original construct is in the genome.

Plant Regeneration

Clones that were sequenced and predicted to have lost the expression of the target genes and found to be free of the CRISPR system DNA/RNA were propagated for generation in large quantities and in parallel were differentiated to generate seedlings from which functional assay is performed to test the desired trait.

Solubility Assay

Solubility is determined by measuring galactomannans. An increase in galactomannans content is an indication of increased galacomannans to mannas ratio, therefore increased solubility. Galactomannans can be measured indirectly by sequential enzymatic reactions involving β-mannanase, α-galactosidase and β-galactose dehydrogenase and release of D-galactonic acid and NADH. The release of NADH is assayed spectrophotometrically at 340 nm (McCleary B. V., 1981, An Enzymic Technique for The Quantitation of Galactomannans in Guar Seeds, Lebensmittel-Wissenschaft & Technologie, 14, 56-59).

TABLE 4
Primers

| Primer | |
|---|---|
| ID | Primer sequence |
| 96 | TCCAGTCCTACTTTATGATTGAAAA SEQ ID NO: 43 |
| 97 | CATCAACCAAAATTGAGACCAA SEQ ID NO: 44 |
| 98 | TCATTTTGGATTTTGGCACA SEQ ID NO: 45 |
| 99 | TTTCCTTGGGGCTTATGTTG SEQ ID NO: 46 |
| 114 | ACACTGGATGGCACGTTGTA SEQ ID NO: 47 |
| 115 | AGACCTACCCCAGACCCAGT SEQ ID NO: 48 |
| 116 | TGAGGAGATGGTATTGGGAGA SEQ ID NO: 49 |
| 117 | CCCCTTACCTCTCTCGTCTCT SEQ ID NO: 50 |
| 118 | CCTGTCGAATGTCCAAGGAA SEQ ID NO: 51 |
| 119 | GTGCATGCTCCTCAAGACAA SEQ ID NO: 52 |
| 120 | GAATGGAAGTGGGACCATGT SEQ ID NO: 53 |
| 121 | GCTTCCCATCCAAATTAAACC/SEQ ID NO: 54 |

### Example 2

### FACS Enrichment and Isolation of Non-Transgenic Genome Edited Protoplasts

To assess that the CRISPR/Cas9 complex and sgRNAs are functional when transfected to coffee protoplasts, 4 reporter-sensor plasmids were prepared that consisted of a red fluorescent marker (dsRed), Cas9, a GFP fluorescent marker and sgRNAs targeting GFP in one vector (see FIG. 2). Sensor 1 and 3 have the same sgRNA but different U6 promoters and sensor 2 and 4 have the same sgRNA but different U6 promoters. All 4 plasmids were delivered independently into protoplasts derived from Coffea canephora (FIG. 2) and confirmed Cas9 activity in these protoplasts by measuring the ratio of green versus red protoplasts using FACS. Evidence of genome editing of the GFP marker is shown as a reduction of the green versus red ratio when compared to the control plasmid, which only lacks the sgRNAs. As shown in FIG. 2, all versions of the reporter-sensor plasmid indicate that Cas9 is active in coffee and leads to positive editing thereby specifically reducing the signal of the GFP marker.

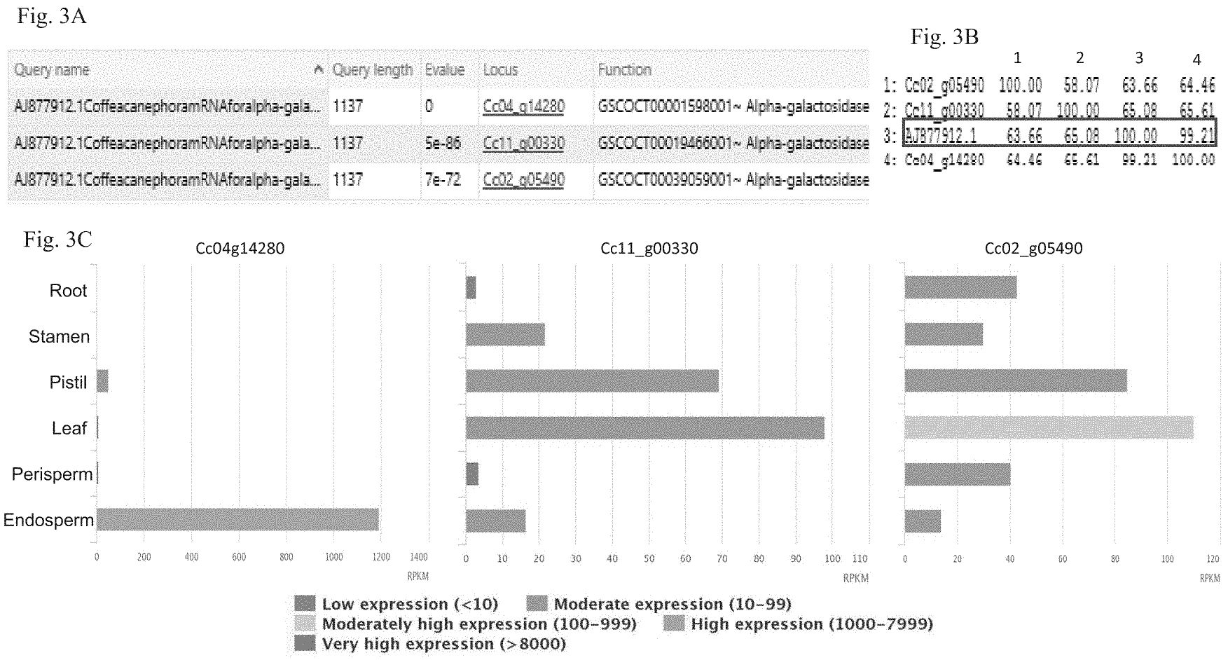

Next, the alpha D galactosidase genes within the coffee genome were identified by blastn using as query the sequence with accession number AJ887712.1 submitted and described by Marraccini et al., 2005, supra. As seen in FIG. 3A, 3 genes were retrieved that correspond to the alpha D galactosidase in the published genome of C. canephora. The percentage of identity of 99.2% indicates that Cc04_914280 is the homolog of AJ887712.1 (FIG. 3B), which has been biochemically and molecularly characterized as alpha D galactosidase in coffee beans. The additional 2 sequences exhibit a lower similarity between 60-65% (FIG. 3B).

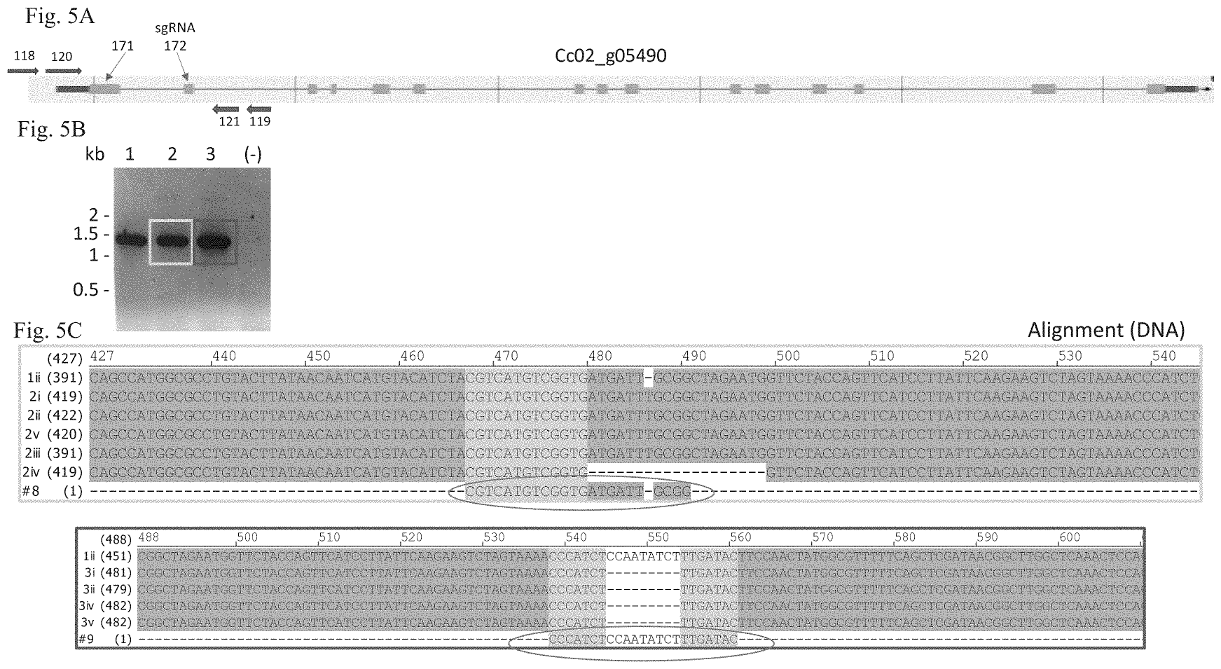

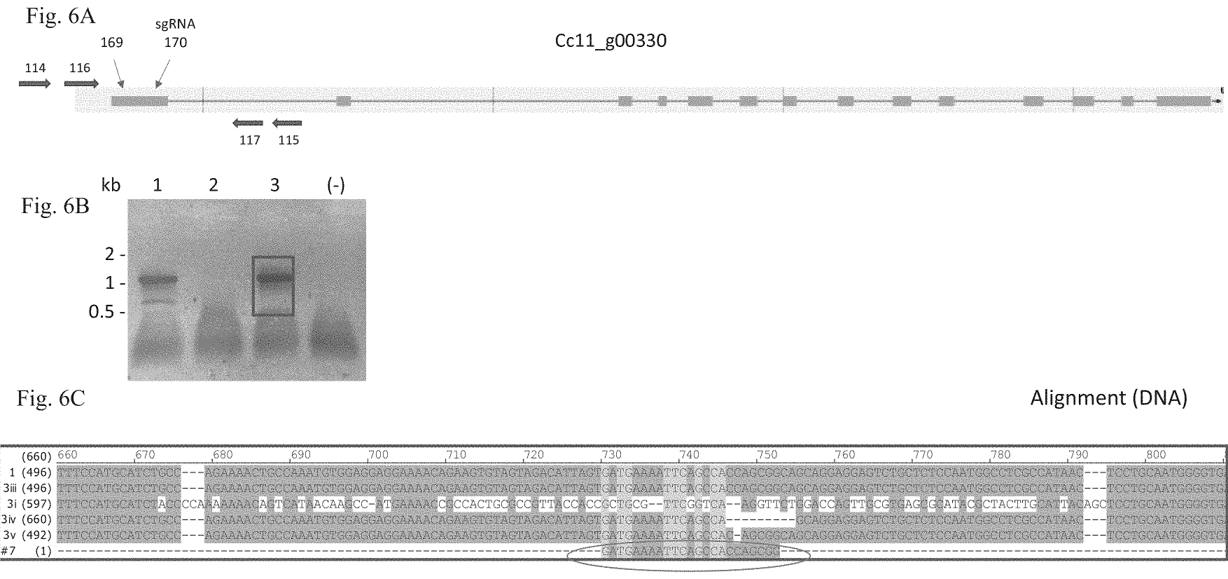

To get some insights into the roles of these genes, the publicly available expression data was retrieved for these 3 candidate genes (Cc04_g14280, Cc11_g00330, and Cc02_g05490) (FIG. 3C). The RPKM data of each gene from the coffee genome database indicates that Cc04_g14280 is highly expressed in endosperm emphasizing its significance in the solubility of coffee beans (FIG. 3C). However, given that the other two genes still show moderate expression not only in endosperm but in other tissues, it was decided to design sgRNAs targeting all genes. Cc04_g14280 was targeted with a pair of unique and specific sgRNAs that are on exon 2 and 3 as indicated in FIG. 4A. This region was selected because it is the closest to the 5′UTR for which a PAM motif could be identified. To design the sgRNA pair, CRISPR RGEN Tools (www(dot)rgenome(dot)net/) were used. CRISPR RGEN employs an algorithm that designs the sgRNA sequences according to their quality and lack of off-target activity in a given genome (FIGS. 9A-C). The two sgRNA shown were cloned into a plasmid which contained mCherry, Cas 9, and two sgRNAs driven by a U6 pol 3 promoter. In a similar manner, sgRNAs were designed and cloned into plasmids for protoplasts transfections for the two additional candidate genes Cc02_g05490 and Cc11_g00330 (FIGS. 5A-C; FIGS. 6A-C).

Next, the CRISPR/Cas9 complex and sgRNAs that target the gene Cc04_g14280 were transformed (as described above using PEG) into coffee protoplasts line FRT06 and enriched for cells that carry such complex by fluorescence-activated cell sorting (FACS). Using the mCherry marker, transfected coffee cells that transiently express the fluorescent protein, Cas9 and the sgRNA were separated, sorted and collected mCherry-positive coffee protoplasts at 3 days post transfection (dpt). DNA was extracted from 5000 sorted protoplasts (Qiagen Plant Dneasy extraction kit) at 6 dpt. Nested PCR was performed for increased sensitivity using primers shown in FIG. 4A. PCR 1 consisted of 20 cycles using Phusion polymerase, 2 ul DNA template, forward and reverse1 primer with an annealing temperature of 60 degrees and extension time of 60 seconds. No additives were added in addition to the HF buffer supplied in the kit. PCR2 was performed using 20 cycles with 1 ul of DNA template from PCR1 and the forward and reverse2 primers. The agarose gel indicates a deletion has occurred in the target gene of around 250 bp (FIG. 4B).

PCR products 1 and 2 (FIG. 4B) were cloned into pGEM-T following manufacture's protocols. Five separate colonies of each ligation were screened by sequencing. The alignments were performed using Vector NTI align X program. As shown in FIG. 4C, the sequence from PCR product 1 was the same as WT while all 5 colonies from PCR product 2 showed a deletion of 239 bp situated between the two sgRNA target sites, 3 bp upstream from the PAM site. With the sequenced clones, we predicted the longest peptide sequence for both clones from lane 1 (non targeting sgRNA plasmid pDK2029) and lane 2 (sgRNAs targeting Cc04_g14280, plasmid pDK2030). The 239 bp deletion induced an early stop codon as indicated by the red box (FIGS. 4D-E).

Following the same procedure described above for candidate gene Cc04_g14280, the two additional candidate genes Cc02_g05490 and Cc11_g00330 were targeted with specific sgRNAs as indicated in FIG. 5A and FIG. 6A, respectively. For both genes, Cc02_g05490 and Cc11_g00330, only one sgRNA targeting each gene was cloned into the transfection vectors. Therefore, it was expected that any gene-editing events were not visible in an agarose gel. FIG. 5B and FIG. 6B show amplification of the targeted region for genes Cc02_g05490 and Cc11_g00330, respectively. The bands shown in FIG. 5B and FIG. 6B were cloned into pGEM-T and sequenced. Alignments with wild type sequence performed with Vector NTI align X program showed the presence of indels along candidates genes Cc02_g05490 and Cc11_g00330 in FIG. 5C and FIG. 6C, respectively.

In parallel, additional mCherry protoplasts that were advanced in the protoplasts regeneration pipeline were sorted. Briefly, sorted protoplasts were plated at high dilution in liquid medium to allow colony formation for 28-35 days. Colonies were picked, grown and split into two aliquots. One aliquot was used for DNA extraction and genome editing (GE) testing and CRISPR DNA-free testing, while the others were kept in culture until their status was verified. Only the ones clearly showing to be GE and CRISPR DNA-free were selected forward.

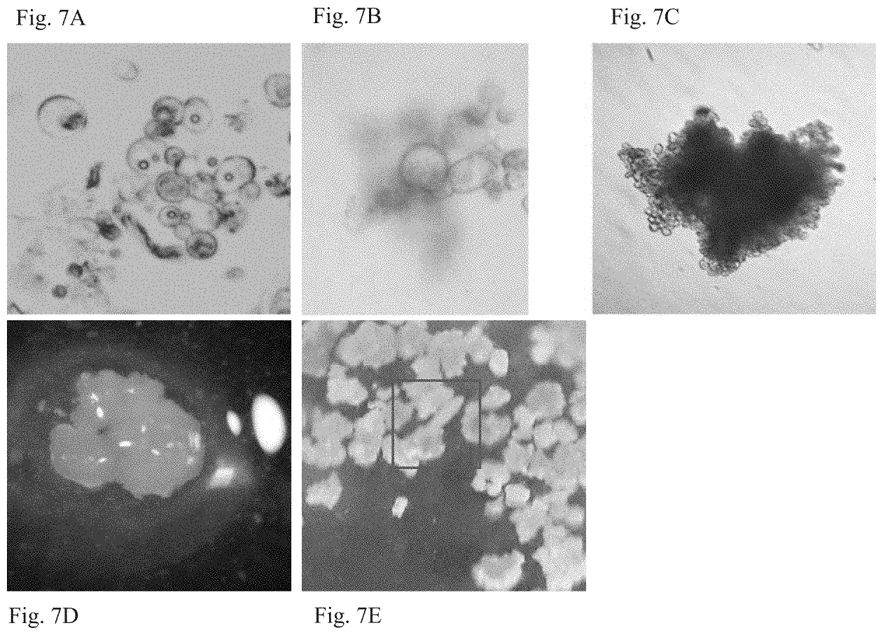

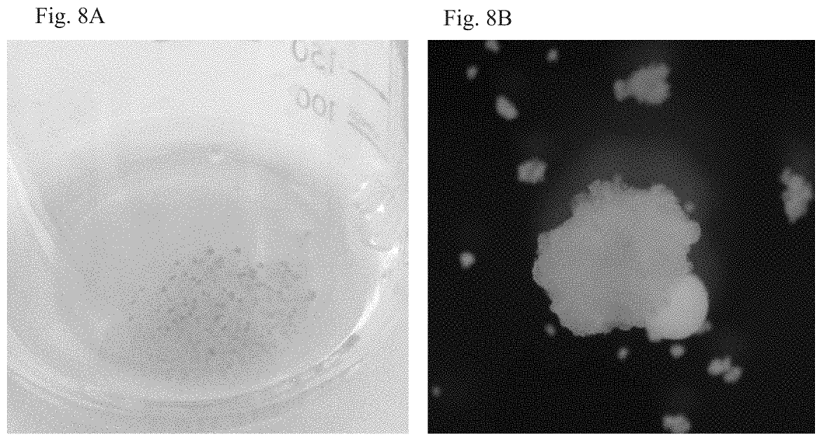

After 20 days in the dark (from splitting for GE analysis, i.e., 60 days, hence 80 days in total), the colonies were transferred to the same medium but with reduced glucose (0.46 M) and 0.4% agarose and incubated at a low light intensity. After six weeks agarose was cut into slices and placed on protoplast culture medium with 0.31 M glucose and 0.2% gelrite. After one month, protocolonies (or calli) were subcultured into regeneration media (half strength MS+B5 vitamins, 20 g/l sucrose). (FIGS. 7A-E; FIGS. 8A-B).

Used are the sgRNA sequences and constructs shown in FIGS. 9A-C.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. (canceled)

2. A method of increasing extractability of solids from coffee beans, the method comprising:
(a) subjecting a coffee plant cell to a DNA editing agent directed at a nucleic acid sequence encoding alpha-D-galactosidase to result in a loss of function mutation in said nucleic acid sequence encoding said alpha-D-galactosidase; and
(b) regenerating a plant from said plant cell.

3. The method of claim 2 further comprising harvesting beans from said plant.

4-6. (canceled)

7. The method of claim 2, wherein said mutation is selected from the group consisting of a deletion, an insertion, an insertion/deletion (Indel) and a substitution.

8. The method of claim 2, wherein said coffee plant is from a species Coffea arabica or Coffea canephora.

9. (canceled)

10. The method of claim 2, wherein said subjecting is to a nucleic acid construct encoding said DNA editing agent.

11-12. (canceled)

13. The method of claim 2, wherein said DNA editing agent is of a DNA editing system selected from the group consisting of meganucleases, Zinc finger nucleases (ZFNs), transcription-activator like effector nucleases (TALENs) and CRISPR-Cas.

14. The method of claim 2, wherein said DNA editing agent is of a DNA editing system comprising CRISPR-Cas.

15. The method of claim 2, wherein said nucleic acid sequence encoding alpha-D-galactosidase is as set forth in SEQ ID NO: 4.

16. The method of claim 2, wherein said nucleic acid sequence encoding alpha-D-galactosidase is selected from the group consisting of SEQ ID NOs: 2-4.

17-18. (canceled)

19. The method of claim 2, wherein said DNA editing agent is directed at nucleic acid coordinates within exon 1, 2, 3, 4 and/or 5 of a nucleic acid sequence encoding said alpha-D-galactosidase.

20. The method of claim 2, wherein said DNA editing agent comprises a nucleic acid sequence at least 99% identical to a nucleic acid sequence selected from the group consisting of SEQ ID NO: 38-41.

21. The method of claim 2, wherein said DNA editing agent comprises a nucleic acid sequence at least 99% identical to a nucleic acid sequence selected from the group consisting of SEQ ID NO: 9-11 and 37.

22. The method of claim 2, wherein said DNA editing agent comprises a nucleic acid sequence selected from the group consisting of SEQ ID NO: 38-41 or a nucleic acid sequence selected from the group consisting of SEQ ID NOs: 9-11 and 37.

23. (canceled)

24. The method of claim 2, wherein said DNA editing agent is directed to a plurality of alpha-D-galactosidase genes.

25. The method of claim 24, wherein said plurality of alpha-D-galactosidase genes are selected from the group consisting of SEQ ID NOs: 2-4.

26-39. (canceled)

40. The method of claim 2, wherein the plant is non-transgenic.

41. A nucleic acid construct comprising a nucleic acid sequence encoding a DNA editing agent directed at coffee alpha-D-galactosidase being operably linked to a plant promoter.

42. The nucleic acid construct of claim 41, wherein said DNA editing agent is directed to a plurality of alpha-D-galactosidase genes, optionally wherein said plurality of alpha-D-galactosidase genes are selected from the group consisting of SEQ ID NOs: 2-4.

43. A coffee plant, or a plant part thereof, comprising a genome comprising a loss of function mutation in a nucleic acid sequence encoding alpha-D-galactosidase, wherein the plant is non-transgenic, optionally wherein the plant part is a bean.

44. The coffee plant of claim 43, wherein said nucleic acid sequence encoding alpha-D-galactosidase is selected from the group consisting of SEQ ID NOs: 2-4, optionally wherein said nucleic acid sequence encoding alpha-D-galactosidase is as set forth in SEQ ID NO: 4.


SEQUENCE LISTING (54 sequences)

SEQ ID NO: 1 - DNA, 9163 bp, Coffea canephora
ggagttcatttatcattcaaattctgtcaatcaaactcttgctttctcagctcaacactaaaccataccacttctggggggctctgctccacaaagcagtggcaattgagttgattgatcaacaccaatttaccatggccgctgcttattactaccttttttctagtaaaaaaagccaccaaaagctggtgcttcgagcttcgttattgatgtttttatgtttcttggcggttgaaaacgttggtgcttccgctcgccggatggtgaagtctccaggaaccgaggattacactcgcaggagccttttagcaaatgggcttggtctaacacctccgatggggtacgtttttagttgttatttaatcttacgttcttgttttgtttctaatgaattgatattaatgttagttaattagtacttaattagcagttttgtggtttggataaaagtgaagagaataaaatctctgaggtagtaatctggattatggaagaagagtatcccactattgcatgttgaagttgaagggatttattcacaaaaatgtggaaaatagttttccgcatagacagttaaatccggcattatgcactttttggcctgtttgcttgttttagtatatttttaactcttctattttccagctgttctggatattattaggacaaaaaaaaaacctttcttttaatatttttctcctttttaatgtagacgacctttcccaaagaaaaaaattgtttacaaaaatgagtaatatttgtgccataaattgttggaattggttaggttcttgaaaagctttatccatctgtctagatccagtaggtggcctcacgctgccaaagtgtcggcaaacgacaatttatcgtacggtggtattttgctgaatctacaaattaagatacattccaagtaattaagggtttttctaattaagacgtgtttagttaagggttttttaataggtgtttagtggacggttagtagtgtacttaatttataccaaacgtactatatgcatgaagttacatttaatattagtttctcctaaagattaccatctcaaatccatgatagtaagtacgagttagagttagatattattagttagtttttcaaggtaaatgagatgatatgatgttctcatcatgttttaattttttctaattgcatcgcatgcagcaccgtataattaacacttgtaatcgaaatatagaaatatagaattagacgccacaaaaagaagatacaagtatagatacacgtactttatatatatatatatatatatataaagacactactattttaagagataaggtctaacctcattctcgtatttttggtttcctttttgtcatttataaaacagatagcggtcccaatcaagcctccatcgtctgaaagtagtatataattaagaaaaaatataaattaaatcatgagctatttgcataaattacaattaagtttagctcaacgagctttatggatgtattaattagttggcacattaaattactcttagaaaacatcactggttttcatttcatgttaaactaacggcttttatacaaaattttaggaatgaaaccttttgtgtatacccaataaattggaggagaaatgtctgtcatttttatttaattagagattggatgcaagcatattaatttttgttaggtagttggtatggcattgcgggtcattactttctctttgactttgacccaatcggtgtatgtcactttgcttgtatccaattcccatatattccttccaagtttaggtagctacaactcccatgacatttgacaccaatcaattaaattatgacgattatttccctttcccactatctaacatttacaagtcacaaggacaatagggttcacttattgcattgctgtgttcatcaaattaagttgggagacggtgcaaaattgagtttttgacataaatgttaataggcttagggcattcattatccctaaaaaaatgaaaaaaaaaaatgatacagaagcccattaactagccaaaactttggttagaaaagaatgcaaagagagaagtgaaagataaggtagaaaagacacaacaaattgaggatggttcgttagttctttcatagttagttcaccaatgaagaaagcaggaaccactaaggaactattgcggttggtaggggcaatggaggaaggcgaattgcattcgacaattgacggcaaagactagcccggtaagttttgggccgatgtcaaccatcaaacatggttttttaaaaacaaactaacttcaatgaaggttgtatggtcaaaacgatgtcaaactcatgcagaccgatcttagccaacaatttgtccttcatgattgcacataaattttgaatcaaaaccccccaacttatttttaaaaaataaaagaaaataaattaaatttgtatggccaagtgcgaccaaataatcatatctatttattgggctcctcaaggtgcatagatgccttacatccattattttgaaaatcggatcagattgatttatttgaccgatcgaatcatgaaactatcatgcctctaattcggttcaatgattaatctaaaagctaattgaaccagtcaaactcagcaactagtaaaaattgaaaggttgaatcgaatttcgttaattttttattttttaaaacaaatattttagttttgttcaatttttttactaataaattaaataaaaaataattaaacttttaaaccaaaaatgtaccgatcagaccggttaaaccgcaaatcgaattcgattcactttcctgtccacatttaaaaacatttccttgcatgggcatagggtttattctgggggtaggggatgctggtgaagatgttaccagacatcgatctcagataatcactgacctcgattggattttgtacatatatactggtgctctacatgtccgtggataggcccgtgaaatgggtttattcaatgaaacataaaacaatatgtcagatttttgccaggacaatgtgtcagatttcgttgtcagtatctcagtactgtgctaagtaaagatcagatgaacatattcggctatgatttattttatgcaagaactaatgtaagccattaaagcgtaggttcttactgcattagaaatatcctcagctagaatgtgttatatatatatatatatatataactttcagggaaaaaaggaataaaagttccagtcctactttatgattgaaaaaatagaataaaagttccaattaatcctagtttttgattaagaaaagaaatctaatgagctaagagtatgatatgttaaaaagaatacacaaatggcgattatctcatttggaggttcgatttgggcaaggaatatcctcatttcattttggattttggcacagatccagcacattatgcttatgtctgtgcatatataaatatggaaagatacacttatgaatctaaatatgaaataccaagtaatcctagtgaaatttctaagcatttactttgtgtaaaatgcaattatcaggtggaacagctggaatcatttccgttgtaatcttgatgagaaattgatcagggaaacaggtaagctttttgtggaggaccccgaaactttgactcaaattaaagggccttttgtatataatggccaacagtccatttttaattactattactcactaactactttgctagctaacttgccaagtttataagactttttcctttcactggttttaatttgatcaacagccgatgcaatggtatcaaaggggcttgctgcactgggatataagtacatcaatcttggtacgtagaaaaaagagtggaaagtcaaaaggatgatgcttttcttttttctgtatcagctcatttactaatgggttacgatttttagcaaaattaattggaaactaatcttttcgtggattgagagaagaaagagttttaacactagtgagataggatgcatacaaaatgtgtaaatcgaaatgtcaaaatgaaaaagcgattacagacatatccaaaactattaatgtgaaaaaaaaccttttgcattgataggaaaaagtaatccattaattcattgttagaatattattgaatgtggaccttttttttatcaaataaaatcataaattgctaaaattttctacttggatatttggtagatgactgttgggcagaacttaacagagattcacaggtatatactccttctcatcactctaagatgaactatatggctcacataacactactatagtagataattagcatcagaaagagaactttttccatagtatagcttcttgggtgaggctgaaatatgagctatgtgttgcggtgcaggggaatttggttcctaaaggttcaacattcccatcagggatcaaagccttagcggattatgttcacagcaaaggcctaaagcttggaatttactctgatgcggggtaaaacttgaactttaccttagcttctactaatggttaccagtttactaccagaatacaaattaaatttcatcgagctagcatagcactagcatggtaattaatgttctaattttgtaatttgatgatgcagaactcagacatgtagtaaaactatgccaggttcattaggacacgaagaacaagatgccaaaacctttgcttcatgggtatgtacatactagttacttctattgattggcgcatgtttcgttgtgttttctgtcaatagtgcttgtttaatgatatatttctgtatttatgagaattaccatcacaaatttgcttttaatttttccccctatcactaagctttatctccaaatttaacttgtaagagcattaatttgcttaaattattctactacctgcctatttggcataattgtgtttctgaattcaaaatttttaattctctttctatcttaccctattggtattagggggtagattacttaaagtatgacaactgtaacaacaacaacataagccccaaggaaaggtatgtattatgtacaaactgctctccaactaaatggtactctaacgaagcaattagtgtcaaaatttggtctcaattttggttgatgaccaattgaaccaataatttgtatctatagtacccttttatctagtgttttgtccttgtggtgaaataggtatccaatcatgagtaaagcattgttgaactctggaaggtccatatttttctctctatgtgaatggtgagtcttggttttatggacctcattcggtcagttgtaattcgacataaaatgctatattagcaaaatgggggttcaattattttggatgaatagccaagatcatcaaaataatggtcttaaattctttctcagctgattaattccgctgtgtatgatatcaggggagaggaagatccagcaacatgggcaaaagaagttggaaacagttggagaaccactggagatatagatgacagttggagtaggtaataatactacctaggacatctcttaacttgcttcttgtttgagttgtttgatatatatatatatataattttgttgcaaatggatgatcaattgctacaacttctagtaattaatctggaatgtttttaacaatgctccttgaaaaagggcaaaaatatttctagcaatgcatcccgagaataaaaaagcatattgcatttttttacgttacccaaaaaaaagaccatatatgatacatttttgctaaacaacacaagtgaatattgtaaaattttcatcactacaaattaggagctgatgaaatttcaaataaagaatgtatagaaaagatataaaattaaacattaagaccaattttttttgtattattaattttttggcttggttgggatgcgcagcatgacttctcgggcagatatgaacgacaaatgggcatcttatgctggtcccggtggatggaatggtatttatctcactttttgtttaataataattttcatttgtgcaaatgacaaatttatcactctatatttcaatattatcctgacaatggctacttcacaagtactaaccatgaaatacaatactataaaaccatacaatcaaatttatcttgctttgtcggaacaggatggtatggtgaaaggaattttaaataggagattctgaattcaaaattttctatttattaaaaaaaatctttctttgtttattagtacaagtattgatagcttgctttgtgtgtgtaaaagtacataataaatgggtatttttgaaaacaaaactaagtcattgctatttaggagtcatttagtctttctatgagtaacatgtacatgtcatgtcagcaaaatgaaagagtaattgggactaattatttactgattatattggattcaagaaaattcaatactatagtgggaagattgatgcaattgagtattcatgtggcaaactcatgaaattgtactttttcgtgggggaatttgcaattaagcctacttttaaattttgcagaagtgtagacaggaaaacacgtccttacattggtattcccaaattaatattttttgaaggttattggatttcacatattcttacacaaagacatgcacatgcattaactcacggatgagaaaactaacacaacgtggcatcgtacacttgttgaaaacttaaggccatatttgaattgctcttttctagaaaaacatttaacctttttttttaaatattctttttacatatttgtcaatcactttttaactgtccatacatccaattctaaaaaagtgattcagtaattttttccctaaaaaactctcgaaaatttacaatccaaaaaatctctaattgtttcagatcctgacatgttggaggtgggaaatggaggcatgactacaacggaatatcgatcccatttcagcatttgggcattagcaaaagtatgttcactaataagtgagaagatgctattactttttttttttctccttttttctaggtatatatgggatccactatacactataagaaaattatgatcattaatcaagaacaataatcttgttacagcacaaacacatatagacgtatattatgatgtatatattaaataattgatcatagtgctaatttagatttaattaattgtttggctgtttattaatttatgaattattttgtgcttataaatatccatgaaggcacctctactgattggctgtgacattcgatccatggacggtgcgactttccaactgctaagcaatgcggaagttattgcggttaaccaaggtatggaccaaagaagatatcgatacaagtgcatatattggaccctggactgaattggactgaaatggagttcttggatacttcttaatcagctttaagagacttgaattgattagttatagcttttttttctccatcgacaaaaagagctaaacatacaaatgatgatattctcttttttcacatggcatcttgactaatacattgcaaatcttatctatagataaacttggcgttcaagggaacaaggttaagacttacggagatttggaggtgaatttctgaaacaatctagattgcatgtttgtcccttcatttttcatgcattagtgcccaaaatacctttaaacttaggtgtctcatttgtcaaattattgataagtattaccattttttcttctttccttgatttgtgggaaagaccatgacataattgataaattcaagtgttttgttttgtttggcaaatgcaataattaatggtttctgtttttatgtactatctgtgcaaatattttttgcactggtagttgtaaataaatgccacttgttgaaaattaaattttaaatttaaaattgaattatgtgacataaatacagtatcactcgtgtatacacaaatgatatataaaaaattaatcctaattaataatactaatcagttttctgctaatgctgcaggtttgggctggacctcttagtggaaagagagtagctgtcgctttgtggaatagaggatcttccacggctactattaccgcgtattggtccgacgtaggcctcccgtccacggcagtggttaatgcacgagacttatgggcggtaatacctcaacggttctttaaattcattgggcaacaatcgctattatagatacttttaaactactcataaaattatacttcatttgccaaccagaaagaattaccattaaaatcataatttaagcagtgaaccttaaacccaatccccgtttgcgttgcactccatttcctaaaatagcacttttggaagacaaaagcacttttacatgtttagtgagcatcatttcttgaattcaggagtaaagttttttgccagtaaaccacttttggtgtaaactcaaaattggttattctagagtaatttttgtgtttcaaaaagtaaataattaactttaacaattttattataaattttgaactaaatgaagagataaatatatattagaaattcaaaattacacattattaaatagaaaagaatacttatgcaagtatatacccaaataatattattaaacacttaataagtattttgatcaaaagttctattaattaagtgctttttgataaaccaccgtgacttcaaatgggctcttaggtgactaaatatgttgtcatgtattcaaaattagtacaaaagctaaataaatttttgggattatgattatactaattcaatattgaaattactatttggcattcagcattcaaccgaaaaatcagtcaaaggacaaatctcagctgcagtagatgcccacgattcgaaaatgtatgtcctaaccccacagtgattaacaggagaatgcagaagacaagtgatggttggctctttcaaggatttgattaccttaaagaatttttcacatgttatgaatcaattcaaagcaattatgtgttttgaagagattaagtcaataaatagaaaagttattattgaaaaaacaaacttcatctattatagcaattaactattgtctatctattatttatcatcgactagtatattgtatattctagtttctttccttttctatagtatctaaaacacgctttattttttgtagtatctaaaacacgctttatacaacaaaggaaaagagaacattaagac

SEQ ID NO: 2 - DNA, 1311 bp, Coffea canephora
atggaggacaggaagaagccatcaatttcgtcgcctgctaccaagttctttattgttttgttattcatcttctttttggatattcatggtggcggccattatagtttccatgcatccgccagaaaactgccaaatgtggaggaggaaaacagaagtgtagtagacattattgatgaaaattcagccaccagcggcagcaggaggagtctgctctccaatggcctcgccataactcctgcaatggggtggaatagctggaatcactttgcctgcaacgttagcgaggaacttatcaaagaaacggctgatgcactggtttcaactggcctgtccaagcttggatatcaatatgtgaacatagatgattgctgggcagaaattaaccgtgatgacaagggaaatctagtgcctaagaagtctacttttccttcgggcatgaaagcccttgcagactatatccacagcaagggactcaagttgggaatctactcggatgcagggtattatacttgtagcaagaaaatgccaggttctcttggttacgaggaaaaggatgcaaaggcctttgcatcatggggtatagattatctcaagtatgataactgcaacaccgatggctcgaagccagtcgagagatatcctgtaatgacccatgccctgatgaaagctggccgtcctatatacttctcgctgtgtgaatggggagatatgcaccctgctctatggggaggaaacttaggcaatagctggagaaccacaaatgatataagtgatacttgggacagcatggtctccagagcagacgagaatgaagtatatgcagaatatgcaaggccaggcggctggaacgatcctgacatgcttgaggtgggaaatggaggaatgacaaaaaatgaatatattgtccacttcagtatttgggctatttccaaggctccccttctgattggctgtgacgtaaataatataacaaaagagacaatggaaattcttggcaacgaagaggttattgcagttaaccaagataagtttggtgttcaagctaaaaaggtccgaatgctgggtgatttggaggtatgggctgggccactttcggattacagagtagcagtgctgctcgtgaaccgcagcacaaggcgggactccatcacggcccactgggaagatattgggctgcccctaaagactgttgttactgtaagagatctttggcagcacaagactttgaagaaaaagtttgtgggcagcttaactgctacagtggattatcatgcttccaagatgtatatcttcaccccagataggtcttga

SEQ ID NO: 3 - DNA, 1275 bp, Coffea canephora
atggcgcctgtacttataacaatcatgtacatctacgtcatgtcggtgatgattgcggctagaatggttctaccagttcatccttattcaagaagtctagtaaaacccatctccaatatctttgatacttccaactatggcgtttttcagctcgataacggcttggctcaaactccacagatggggtggaatagctggaatttttttgcttgcaacatcaatgaaacagttatcaaggaaacagcggatgcactgatctccactggtttagctggcctaggttataactacgttaatatagatgattgctggtccagctgggttcgaaactcgaagggtcagttggttcctgatcctaaaactttcccatcaggaatcaaagctcttgcagattatgtgcatgcgaaagggctcaagcttggtatctattctgatgcaggagtttttacttgtcaagttcgacctggatcactataccatgaaaatgatgatgcagctctctttgcatcttgggatgtggattatttaaagtatgacaactgcttcaacttgggtatccagccaaaagaaagatacccgccaatgcgagatgccctaaatgcaactgggcaaaaaatattctattctctttgtgaatggggcgttgatgatcctgctctgtgggctggcaaagttggaaatagctggcgtacaacagatgacatcaatgattcatgggcaagcatgactagtattgctgatctaaatgacaagtgggctgcttatgctggtcctggtggatggaatgaccctgatatgttagaggttgggaatgggggaatgacttaccaggaatatcgagcacattttagcatttgggctttgatgaaggctcctcttttggttggttgtgatgtgagaaatatgatgtctgaaacatttgaaattctgagcaatgaagaggttattgctgtaaatcaagactcacttggggttcagggaaggaaagtttacgtttctggaacagatggatgtgaacaggtttgggctggccctttatctgagcaacgtgtggttgttgttctatggaatcgatgttcaaaagttgcaactattacggctggatggtcagcattgggactcgaatcttcaacccctgtgtctgttagagatttgtggaagcatgaagttgttgcggataacagggtggcttcattaagtgctcaagttgaagctcacgcatgtgaaatgttcattttaactcctcagactactactaactctcagattctgtaa

SEQ ID NO: 4 - DNA, 1137 bp, Coffea canephora
atggtgaagtctccaggaaccgaggattacactcgcaggagccttttagcaaatgggcttggtctaacacctccgatggggtggaacagctggaatcatttccgttgtaatcttgatgagaaattgatcagggaaacagccgatgcaatggtatcaaaggggcttgctgcactgggatataagtacatcaatcttgatgactgttgggcagaacttaacagagattcacaggggaatttggttcctaaaggttcaacattcccatcagggatcaaagccttagcggattatgttcacagcaaaggcctaaagcttggaatttactctgatgcgggaactcagacatgtagtaaaactatgccaggttcattaggacacgaagaacaagatgccaaaacctttgcttcatggggggtagattacttaaagtatgacaactgtaacaacaacaacataagccccaaggaaaggtatccaatcatgagtaaagcattgttgaactctggaaggtccatatttttctctctatgtgaatggggagaggaagatccagcaacatgggcaaaagaagttggaaacagttggagaaccactggagatatagatgacagttggagtagcatgacttctcgggcagatatgaacgacaaatgggcatcttatgctggtcccggtggatggaatgatcctgacatgttggaggtgggaaatggaggcatgactacaacggaatatcgatcccatttcagcatttgggcattagcaaaagcacctctactgattggctgtgacattcgatccatggacggtgcgactttccaactgctaagcaatgcggaagttattgcggttaaccaagataaacttggcgttcaagggaacaaggttaagacttacggagatttggaggtttgggctggacctcttagtggaaagagagtagctgtcgctttgtggaatagaggatcttccacggctactattaccgcgtattggtccgacgtaggcctcccgtccacggcagtggttaatgcacgagacttatgggcgcattcaaccgaaaaatcagtcaaaggacaaatctcagctgcagtagatgcccacgattcgaaaatgtatgtcctaaccccacagtga

SEQ ID NO: 5 - DNA, 1137 bp, Coffea canephora
atggtgaagtctccaggaaccgaggattacactcgcaggagccttttagcaaatgggcttggtctaacacctccgatggggtggaacagccgcaatcatttccgttgtaatcttgatgagaaattgatcagggaaacagccgatgcaatggtatcaaaggggcttgctgcactgggatataagtacatcaatcttgatgactgttgggcagaacttaacagagattcacaggggaatttggttcctaaaggttcaacattcccatcagggatcaaagccttagcggattatgttcacagcaaaggcctaaagcttggaatttactctgatgcgggaactcagacatgtagtaaaactatgccaggttcattaggcaacgaagaacaagatgccaaaacctttgcttcatggggggttgattacttaaagtatgacaactgtaacaacaacaacataagccccaaggaaaggtatccaatcatgagtaaagcattgttgaactctggaaggtccatatttttctctctatgtgaatggggagaggaagatccagcaacatgggcaaaagaagttggaaacagttggagaaccactggagatatagatgacagttggagtagcatgacttctcgggcagatatgaacgacaaatgggcatcttatgctggtcccggtggatggaatgatcctgacatgttggaggtgggaaatggaggcatgactacaacggaatatcgatcccatttcagcatttgggcattagcaaaagcacctctactgattggctgtgacattcgatccatggacggtgcgactttccaactgctaagcaatgcggaagttattgcggttaaccaagataaacttggcgttcaagggaacaaggttaagacttacggagatttggaggtttgggctggacctcttagtggaaagagagtagctgtcgctttgtggaatagaggatcttccacggctactattaccgcgtattggtccgacgtaggcctcccgtccacggctgtggttaatgcacgagacttatgggcgcattcaaccgaaaaatcagtcaaaggacaaatctcagctgcagcagatgctcacgattcgaaaatgtatgtcctaaccccacagtga

SEQ ID NO: 6 - DNA, 1442 bp, Coffea  arabica
tgctccacaaagcagtggcaattgagttgattgatcaacaccaatttaccatggccgctgcttattactaccttttttctagtaaaaaagccacccaaaagctggtgctccgagcttcgttattgatgcttttatgtttcttgacggttgaaaacgttggtgcttccgctcgccggatggtgaagtctccaggaacagaggattacactcgcaggagccttttagcaaatgggcttggtctaacaccaccgatggggtggaacagctggaatcatttcagttgtaatcttgatgagaaattgatcagggaaacagccgatgcaatggcatcaaaggggcttgctgcactgggatataagtacatcaatcttgatgactgttgggcagaacttaacagagattcacaggggaatttggttcctaaaggttcaacattcccatcagggatcaaagccttagcagattatgttcacagcaaaggcctaaagcttggaatttactctgatgctggaactcagacatgtagtaaaactatgccaggttcattaggacacgaagaacaagatgccaaaacctttgcttcatggggggttgattacttaaagtatgacaactgtaacgacaacaacataagccccaaggaaaggtatccaatcatgagtaaagcattgttgaactctggaaggtccatatttttctctctatgtgaatggggagatgaagatccagcaacatgggcaaaagaagttggaaacagttggagaaccactggagatatagatgacagttggagtagcatgacttctcgggcagatatgaacgacaaatgggcatcttatgctggtcccggtggatggaatgatcctgacatgttggaggtgggaaatggaggcatgactacaacggaatatcgatcccatttcagcatttgggcattagcaaaagcacctctactgattggctgtgacattcgatccattgacggtgcgactttccaactgttaagcaatgcggaagttattgcggttaaccaagataaacttggcgttcaagggaaaaaggttaagacttacggagatttggaggtgtgggctggacctcttagtggaaagagagtagctgtcgctttgtggaatagaggatcttccacggctactattaccgcgtattggtccgacgtaggcctcccgtccacggcagtggttaatgcacgagacttatgggcgcattcaaccgaaaaatcagtcaaaggacaaatctcagctgcagtagatgcccacgattcgaaaatgtatgtcctaaccccacagtgattaacaggagaatgcagaagacaagtgatggttggctctttcaaggatttgattaccttaaagaatttttcacatgttatgaatcaattcaaagcaattatgtgttttgaagagattaagtcaataaat

SEQ ID NO: 7 - DNA, 23 bp, Artificial sequence
  sgRNA sequence
ggtgaagtctccaggaaccgagg

SEQ ID NO: 8 - DNA, 23 bp, Artificial sequence
  sgRNA sequence
gcttggtctaacacctccgatgg

SEQ ID NO: 9 - DNA, 23 bp, Artificial sequence
  sgRNA sequence
atttctcatcaagattacaacgg

SEQ ID NO: 10 - DNA, 23 bp, Artificial sequence
  sgRNA sequence
tcaaaggggcttgctgcactggg

SEQ ID NO: 11 - DNA, 23 bp, Artificial sequence
  sgRNA sequence
gatgggaatgttgaacctttagg

SEQ ID NO: 12 - DNA, 23 bp, Artificial sequence
  sgRNA sequence
cagagtaaattccaagctttagg

SEQ ID NO: 13 - DNA, 20 bp, Artificial sequence
  sgRNA sequence
ggtgaagtctccaggaaccg

SEQ ID NO: 14 - DNA, 20 bp, Artificial sequence
  sgRNA sequence
gcttggtctaacacctccga

SEQ ID NO: 15 - protein, 42 aa, Artificial sequence
  RVD sequences
HDHDNINHNHNINIHDHDNHNINHNHNINGNGNIHDNIHDNG

SEQ ID NO: 16 - protein, 34 aa, Artificial sequence
  RVD sequences
NIHDNIHDNGHDNHHDNINHNHNINHHDHDNGNG

SEQ ID NO: 17 - protein, 36 aa, Artificial sequence
  RVD sequences
NIHDNIHDNGHDNHHDNINHNHNINHHDHDNGNGNG

SEQ ID NO: 18 - protein, 32 aa, Artificial sequence
  RVD sequences
NIHDNIHDNGHDNHHDNINHNHNINHHDHDNG

SEQ ID NO: 19 - protein, 38 aa, Artificial sequence
  RVD sequences
NIHDNIHDNGHDNHHDNINHNHNINHHDHDNGNGNGNG

SEQ ID NO: 20 - protein, 52 aa, Artificial sequence
  RVD sequences
NIHDNIHDNGHDNHHDNINHNHNINHHDHDNGNGNGNGNINHHDNININING

SEQ ID NO: 21 - protein, 42 aa, Artificial sequence
  RVD sequences
HDNHHDNINHNHNINHHDHDNGNGNGNGNINHHDNININING

SEQ ID NO: 22 - protein, 50 aa, Artificial sequence
  RVD sequences
NINHHDNINININGNHNHNHHDNGNGNHNHNGHDNGNINIHDNIHDHDNG

SEQ ID NO: 23 - protein, 60 aa, Artificial sequence
  RVD sequences
NINHHDNINININGNHNHNHHDNGNGNHNHNGHDNGNINIHDNIHDHDNGHDHDNHNING

SEQ ID NO: 24 - protein, 34 aa, Artificial sequence
  RVD sequences
NHNHNGHDNGNINIHDNIHDHDNGHDHDNHNING

SEQ ID NO: 25 - protein, 36 aa, Artificial sequence
  RVD sequences
NHNHNINIHDNINHHDHDNHHDNININGHDNINGNG

SEQ ID NO: 26 - DNA, 22 bp, Artificial sequence
  TALEN target sequences
tccaggaaccgaggattacact

SEQ ID NO: 27 - DNA, 18 bp, Artificial sequence
  TALEN target sequences
tacactcgcaggagcctt

SEQ ID NO: 28 - DNA, 19 bp, Artificial sequence
  TALEN target sequences
tacactcgcaggagccttt

SEQ ID NO: 29 - DNA, 17 bp, Artificial sequence
  TALEN target sequences
tacactcgcaggagcct

SEQ ID NO: 30 - DNA, 20 bp, Artificial sequence
  TALEN target sequences
tacactcgcaggagcctttt

SEQ ID NO: 31 - DNA, 27 bp, Artificial sequence
  TALEN target sequences
tacactcgcaggagccttttagcaaat

SEQ ID NO: 32 - DNA, 22 bp, Artificial sequence
  TALEN target sequences
tcgcaggagccttttagcaaat

SEQ ID NO: 33 - DNA, 26 bp, Artificial sequence
  TALEN target sequences
tagcaaatgggcttggtctaacacct

SEQ ID NO: 34 - DNA, 31 bp, Artificial sequence
  TALEN target sequences
tagcaaatgggcttggtctaacacctccgat

SEQ ID NO: 35 - DNA, 18 bp, Artificial sequence
  TALEN target sequences
tggtctaacacctccgat

SEQ ID NO: 36 - DNA, 19 bp, Artificial sequence
  TALEN target sequences
tggaacagccgcaatcatt

SEQ ID NO: 37 - DNA, 23 bp, Artificial sequence
  sgRNA sequence
acatgtagtaaaactatgccagg

SEQ ID NO: 38 - DNA, 23 bp, Artificial sequence
  sgRNA sequence
cagaaaactgccaaatgtggagg

SEQ ID NO: 39 - DNA, 23 bp, Artificial sequence
  sgRNA sequence
gatgaaaattcagccaccagcgg

SEQ ID NO: 40 - DNA, 23 bp, Artificial sequence
  sgRNA sequence
cgtcatgtcggtgatgattgcgg

SEQ ID NO: 41 - DNA, 23 bp, Artificial sequence
  sgRNA sequence
cccatctccaatatctttgatac

SEQ ID NO: 42 - DNA, 25 bp, Artificial sequence
  Single strand DNA oligonucleotide
tccagtcctactttatgattgaaaa

SEQ ID NO: 43 - DNA, 20 bp, Artificial sequence
  Single strand DNA oligonucleotide
tttccttggggcttatgttg

SEQ ID NO: 44 - DNA, 22 bp, Artificial sequence
  Single strand DNA oligonucleotide
catcaaccaaaattgagaccaa

SEQ ID NO: 45 - DNA, 20 bp, Artificial sequence
  Single strand DNA oligonucleotide
tcattttggattttggcaca

SEQ ID NO: 46 - DNA, 20 bp, Artificial sequence
  Single strand DNA oligonucleotide
tttccttggggcttatgttg

SEQ ID NO: 47 - DNA, 20 bp, Artificial sequence
  Single strand DNA oligonucleotide
acactggatggcacgttgta

SEQ ID NO: 48 - DNA, 20 bp, Artificial sequence
  Single strand DNA oligonucleotide
agacctaccccagacccagt

SEQ ID NO: 49 - DNA, 21 bp, Artificial sequence
  Single strand DNA oligonucleotide
tgaggagatggtattgggaga

SEQ ID NO: 50 - DNA, 21 bp, Artificial sequence
  Single strand DNA oligonucleotide
ccccttacctctctcgtctct

SEQ ID NO: 51 - DNA, 20 bp, Artificial sequence
  Single strand DNA oligonucleotide
cctgtcgaatgtccaaggaa

SEQ ID NO: 52 - DNA, 20 bp, Artificial sequence
  Single strand DNA oligonucleotide
gtgcatgctcctcaagacaa

SEQ ID NO: 53 - DNA, 20 bp, Artificial sequence
  Single strand DNA oligonucleotide
gaatggaagtgggaccatgt

SEQ ID NO: 54 - DNA, 21 bp, Artificial sequence
  Single strand DNA oligonucleotide
gcttcccatccaaattaaacc